(19) United States
(12) Patent Application Publication
(10) Pub. No.: US 2025/0257099 A1
(43) Pub. Date: Aug. 14, 2025

(54) BROAD SPECTRUM INHIBITION OF HUMAN CORONAVIRUSES BY LIPOPEPTIDES DERIVED FROM THE C-TERMINAL HEPTAD REPEAT OF BETACORONAVIRUSES

(71) Applicant: THE TRUSTEES OF COLUMBIA UNIVERSITY IN THE CITY OF NEW YORK, New York, NY (US)

(72) Inventors: Matteo POROTTO, New York, NY (US); Anne MOSCONA, New York, NY (US)

(21) Appl. No.: 19/020,996

(22) Filed: Jan. 14, 2025

Related U.S. Application Data
(63) Continuation of application No. PCT/US2023/070308, filed on Jul. 17, 2023.
(60) Provisional application No. 63/389,513, filed on Jul. 15, 2022.

(51) Int. Cl. C07K 14/005; A61K 38/00; A61K 47/54

(52) U.S. Cl. CPC C07K 14/005; A61K 47/543; A61K 38/00; C12N 2770/20022; C12N 2770/20033

(57) ABSTRACT

Described are lipopeptides that inhibit coronavirus fusion to a host cell. Thus a therapeutic for treating or preventing the common cold is described along with methods of inhibiting and/or treating an alphacoronavirus infection. The lipopeptides comprise a peptide unit comprising an amino acid sequence having a high degree of sequence identity to a sequence from the C-terminal heptad repeat of a betacoronavirus S protein, such as that of SARS-CoV-2.

### CROSS-REFERENCE TO RELATED APPLICATION

This application is a continuation of International Application No. PCT/US2023/070308, filed on Jul. 17, 2023, which claims benefit of U.S. Provisional Application No. 63/389,513 filed Jul. 15, 2022, the contents of which are hereby incorporated by reference.

### GOVERNMENT SUPPORT

This invention was made with government support under grant A1160961 and A1160953 awarded by the National Institutes of Health. The government has certain rights in the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### INCORPORATION-BY-REFERENCE OF MATERIAL ELECTRONICALLY FILED

Incorporated by reference in its entirety herein is a computer-readable nucleotide/amino acid sequence listing submitted and identified as follows: One 6,408-byte XML file named “44010-140” created on Aug. 9, 2023.

### BACKGROUND

Endemic ‘common cold’ human coronaviruses (HCoVs) normally cause relatively mild disease. However, zoonotic introduction of a transmissible coronavirus into an immunologically naive human population can lead to a pandemic scale outbreak associated with life-threatening disease. The current coronavirus disease 2019 (abbreviated “COVID-19”) pandemic is a prime example caused by an introduction of SARS-CoV-2. Like its zoonotic predecessors SARS-CoV and MERS-CoV, SARS-CoV-2 is a betacoronavirus thought to have originated from bats. Human-to-human transmission is now widespread. Thus, newly emerging and reemerging zoonotic infectious diseases, many of which are of viral origin, are a continuous threat.

Over the past two decades, the global effort to meet this challenge has resulted in a growing ability to isolate, identify, and genetically fingerprint the causative agents, often with extraordinary speed. This was illustrated by the severe acute respiratory syndrome outbreak in 2003-2004 (SARS, caused by SARS-CoV), the H1N1 influenza virus pandemic of 2009-2010, the Middle East Respiratory Syndrome outbreak in 2012 (MERS, caused by MERS-CoV), and the most recent COVID-19 pandemic caused by the zoonotic coronavirus SARS-CoV-2. Identification and genetic characterization were translated into rapid development of new therapies, although drug discovery, both by high-throughput screening and rational design, requires information that does not immediately derive from knowledge of the viral genome. Importantly, the resources required for classical drug discovery are not easily mobilized for diseases with limited market potential and/or sporadic outbreaks. In these situations, availability of a specific antiviral treatment for immediate use would be crucial, since, in addition to benefiting the infected individuals, it would help contain the outbreak, and protect the healthcare workers who are at the utmost risk of infection.

Furthermore, vaccines have been demonstrated to reduce disease severity, but they may not always prevent virus transmission. Unfortunately, vaccine coverage is likely to be <100% of the population. Therefore, therapeutic interventions aimed at treating disease and preventing virus transmission are needed to fill a crucial gap in our options to control the pandemic.

### SUMMARY

The inventors have surprisingly discovered that viral diseases other than COVID19 can be treated and virus transmission can be prevented using lipopeptides comprising SARS-CoV2-based peptides. The lipopeptides of the present disclosure comprise peptide units which comprise amino acid sequences having a high degree of sequence identity to a sequence from the C-terminal heptad repeat (HRC) of a coronavirus S protein, such as a SARS-CoV-2 S protein. The lipopeptides of the present disclosure also comprise lipid units, such as cholesterol units. Optionally, the lipopeptides of the present disclosure can comprise linkers, such as polyethylene glycol (PEG) linkers. The PEG linkers can be functionalized, e.g., can include maleimide groups. As an additional or alternative option, the lipopeptides can include oligopeptides, such as GSGSGC or GSGSC, which may be in frame with the amino acid sequences having the high degree of sequence identity with the coronavirus S protein HRC sequences. Such in-frame oligopeptides thus may be considered] further components of the peptide units. Examples of such lipopeptides are described in numbered embodiments 60 to 76. Examples of uses of exemplary lipopeptides are described in numbered embodiments 1 to 59.

Though not to be bound by theory, the peptide units of the lipopeptides may block binding of viral protein sequences to surface receptors, such as ACE2, on mammalian cells, e.g., human cells. This may inhibit a virus's ability to fuse with a cell. The lipid units of the lipopeptides may anchor in the cell membrane and may aid in localization of the peptide units. The linkers and/or oligopeptides may provide conformational flexibility between the peptide units and the lipid units.

The inventors have unexpectedly and surprisingly discovered that lipopeptides comprising peptide units having a high degree of sequence identity to a SARS-CoV-2 S protein HRC sequence can inhibit cellular entry of viruses other than SARS-CoV-2. In some embodiments, the lipopeptides of the present disclosure are used to inhibit cellular entry of other coronaviruses, such as alphacoronaviruses and embecovirus. These coronavirus genera include HCoV 229E, HCoV NL63, HCoV OC43, and HCoV HKU1, infection with which causes common cold. in further embodiments, the lipopeptides of the present disclosure are used to inhibit cellular entry of other viruses, such as measles viruses, Nipah viruses, Ebola viruses, parainfluenza viruses (e.g., human parainfluenza virus type 3), and influenza viruses.

Accordingly, some embodiments of the present disclosure relate to methods of preventing a viral infection in a subject exposed or at risk of exposure to a virus other than SARS-CoV-2, inhibiting an interaction between a viral protein other than SARS-CoV-2 spike protein and cellular ACE2 in a subject, treating a viral disease other than COVID-19 in a subject, reducing the severity of a viral infection other than SARS-CoV-2 in a subject, reducing viral load in a subject of a subject infected with or exposed to a virus other than SARS-CoV-2, preventing disease progression in a subject infected by a virus other than SARS-CoV-2, reducing the duration of an infection by a virus other than SARS-CoV-2, reducing the risk of severe disease or death caused by or ancillary to a viral infection in a subject infected with a virus other than SARS-CoV-2, and/or treating a viral infection in a subject infected by a virus other than SARS-CoV-2. These methods are not mutually exclusive, i.e., methods of preventing a viral infection may also inhibiting an interaction between a viral protein other than SARS-CoV-2 spike protein and cellular ACE2, treat a viral disease other than COVID-19 and/or a viral infection by a virus other than SARS-CoV-2, etc. Examples of such methods are described in numbered embodiments 1 to 59.

In some embodiments, the subject to whom a lipopeptide of the disclosure is a subject at increased risk of infection, e.g., a healthcare worker (e.g., a hospital worker, a physician, a nurse, a home health aide, etc.). In further embodiments, the subject to whom a lipopeptide of the disclosure is administered is at increased risk of serious diseases (e.g., an elderly or immunocompromised individual).

### BRIEF DESCRIPTION OF FIGURES

FIGS. 1A-1C depict the SARS-CoV-2 spike (S) glycoprotein domain architecture and structure and are reproduced from Outlaw et al., mBio. 2020 September-October; 11(5): e01935-20. FIG. 1A is a schematic diagram of SARS-CoV-2 S. The N-terminal domain (NTD), receptor-binding domain (RBD), fusion peptide (FP), N-terminal heptad repeat (HRN), C-terminal heptad repeat (HRC), transmembrane (TM) and cytoplasmic tail (CP) domains are depicted. FIG. 1B shows a pre-fusion conformation of SARS-CoV-2 S (protein data base—PDB—identification code 6VSB). FIG. 1C is a model of post-fusion conformation of SARS-CoV-2 S based on homology with HCoV-229E S (PDB 6B30) and SARS-CoV S (PDB 1WYY).

FIG. 2 depicts exemplary embodiments of the lipopeptides described herein, where a cholesterol unit is directly attached to the peptide or attached with various PEG linkers, and the lipopeptide may be in a monomeric or dimeric form. The figure is reproduced from De Vries et al., Science, 2021, 371(6536): 1379-1382.

FIG. 3A-3D depict exemplary embodiments of the lipopeptides described herein. As shown in FIG. 3A, one embodiment of the lipopeptide comprises a peptide with an acetyl unit at the N-terminus and a 6-residue linker followed by a cholesterol at the C-terminus (X-Chol=PEG₄-Chol). FIG. 3B depicts the chemical structure of the dimeric embodiment with [peptide-PEG₄]₂-chol. FIG. 3C depicts the chemical structure of the embodiment with peptide-PEG₂₄-chol. The peptide unit is as defined in FIG. 3B. FIG. 3D lists the peptide sequences of certain peptide, with the numbering at the top identifying residue positions of D-1 in the native SARS-CoV-2 S protein (top sequence; SEQ ID NO. 1). The two “h” symbols indicate the boundaries of the a-helical segment in the 6HB co-assembly with HRN. D-2 (middle sequence; SEQ ID NO. 2) and D-3 (bottom sequence; SEQ ID NO. 3) are sequences that retain the native HRC side chains that contact the HRN trimer but contain hydrophilic substitutions (in gray) designed to enhance solubility without disturbing binding to the HRN trimer.

FIG. 4 depicts inhibition of cell-cell fusion mediated by SARS-CoV-2 S in 293T cells (solid lines) with by lipopeptides derived from SARS-CoV-2 HRC (“SARS”), MERS-CoV HRC (“MERS”), EK1, or HPIV3 HRC. Toxicity (dotted lines) was determined by MTT assay. The figure is reproduced from Outlaw et al., mBio. 2020 September-October; 11(5): e01935-20.

FIGS. 5A and 5B, in accordance with certain embodiments, show inhibition of coronavirus infection in Vero E6 cells by lipopeptides derived from SARS-CoV-2 HRC (“SARS”), MERS-CoV HRC (“MERS”), EK1, or Human parainfluenza 3 HRC (“HPIV3”). All used peptides have PEG4 and cholesterol moiety. FIG. 5A shows the inhibition of SARS-CoV-2 infection, while FIG. 5B shows the inhibition of MERS-CoV infection. These figures are reproduced Outlaw et al., mBio. 2020 September-October; 11(5): e01935-20.

FIG. 6, in accordance with certain embodiments, compares inhibition of SARS-CoV-2 infection (solid lines) and cell cytotoxicity (dotted lines) by lipopeptides derived from SARS-CoV-2 HRC (“SARS”/D1, D2, and D3), EK1, or HPIV3 HRC (“HPIV3). D1 (red diamond) is superior to EK1 at blocking SARS-CoV-2-mediated fusion (solid lines; left y-axis). D2 (with enhanced solubility) matches the efficacy of D1 at blocking SARS-CoV-2 mediate fusion, while D-3 is slightly inferior. HPIV3 peptide is ineffective. Cell toxicity is minimal for D1 & D2 at IC₉₀ inhibitory concentrations. All the peptides were conjugated to cholesterol. Data are from three separate experiments+/−SE.

FIGS. 7A and 7B compare inhibition of S protein-mediated fusion of SARS-CoV-2 variants by monomeric (FIG. 7B) and dimeric (FIG. 7A) forms of a lipopeptide derived from SARS-CoV-2 HRC and are reproduced from Schmitz et al., mBio. 2022 Jun. 28; 13(3):e0124922. Inhibitory activity was assessed in an assay based on alpha complementation of β-galactosidase (β-Gal) where hACE2 receptor-bearing cells expressing the omega peptide of β-Gal are mixed with cells coexpressing glycoprotein S and the alpha peptide of β-Gal, and cell fusion leads to alpha-omega complementation. Fusion is stopped by lysing the cells, and after addition of the substrate (Tropix Galacto-Star chemiluminescent reporter assay system; Applied Biosystems), luminescence is quantified on a Tecan M1000PRO microplate reader. Fusion between cells expressing SARS-CoV-2 glycoprotein (D614G, alpha, beta, delta, or omicron) and the a subunit of β-galactosidase and human kidney epithelial 293T cells expressing hACE2 receptor and the w subunit of β-galactosidase was assessed in the presence of different dilutions of inhibitory peptide. The resulting luminescence was quantified using a Tecan Infinite M1000PRO reader. Percent inhibition was calculated as the ratio of relative luminescence units in the presence of a specific concentration of inhibitor and the relative luminescence units in the absence of inhibitor and corrected for background luminescence. % inhibition=100×[1−(luminescence at X−background)/(luminescence in the absence of inhibitor−background)]. Values are presented as mean (±standard error of the mean) from three independent experiments.

FIG. 8 compares blocking SARS-CoV-2-mediated fusion by D2 (α-D2), by four α-to-β3 substitutions in the D2 backbone (α/β-F), and by HPIV3 peptide. The variant of D2 with α-to-β3 substitutions is as effective as pure α-D-2 peptide at inhibiting S-mediated fusion.

FIGS. 9A-9D, depict, in accordance with certain embodiments, the ability of SARS-CoV-2 derived cholesterol conjugated peptides to block SARS-CoV-2-GFP viral spread in human airway epithelial cells (HAE). FIG. 9A depicts a schematic of experiment. HAE cells were infected with SARS-CoV-2 (2000 pfu/well) for 90 minutes before addition of SARS-CoV-2 peptide. Fluid was collected from the apical or basolateral surfaces daily for 7 days. FIG. 9B is an exemplary visualization of the spread of fluorescent virus with or without peptide treatment. FIG. 9C compares viral genome copies in apical or basolateral fluids were determined by RT-qPCR at the indicated time points. The median values are represented by horizontal bars, and the detection limit is indicated by dashed lines. FIG. 9D shows infectious virus titers in the apical or basolateral spaces at the indicated time points were determined by plaque assay. The figures are reproduced from Outlaw et al., mBio. 2020 September-October; 11(5): e01935-20.

FIGS. 10A-10C show that dimeric lipopeptides derived from SARS HRC can inhibit coronavirus 229E spread after primary infection. As shown in FIG. 10A, primary human airway epithelial cells grown at an air-liquid interface were infected with HCoV-229E. The cells were treated with the dimeric lipopeptide on the day of the infection and for two days after the infection. Basolateral media and the apical washes were collected on days 1, 3, 5, and 7 post infection, and coronavirus 229E viral genome copies were measured (FIG. 10B). Viral loads were assessed by RT-qPCR from collections on day 3 post infection (FIG. 10C). There were no detectable viral genomes in the basolateral media. Without treatment (only vehicle administered), viral genome increased over time. On the other hand, lipopeptide treatment inhibited viral replication in the human airway tissues.

FIGS. 11A and 11B show that dimeric and monomeric lipopeptides derived from SARS HRC can inhibit spread of common cold coronaviruses after primary infection and have broad-spectrum inhibitory activity against human coronaviruses. As with the experimental design of FIG. 10A, primary human airway epithelial cells grown at an air-liquid interface were infected with a strain of common cold coronavirus. The cells were treated with the lipopeptide (monomeric or dimeric) on the day of the infection and for two days after the infection. Basolateral media and the apical washes were collected on Days 1, 3, 5, and 7 post infection, and viral loads were assessed by RT-qPCR (FIG. 11B). Viral genome copies were measured from collection on day 3 post infection (FIG. 11A). Both dimeric and monomeric lipopeptides reduced the release of the common cold coronaviruses. Though for coronavirus OC43, the monomeric lipopeptide was more potent.

### DETAILED DESCRIPTION

Detailed aspects and applications of the disclosure are described below in the drawings and detailed description of the disclosure. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that the present disclosure may be practiced without these specific details. It should be noted that there are many different and alternative configurations, devices, and technologies to which the disclosed disclosures may be applied. The full scope of the disclosures is not limited to the examples that are described below.

The singular forms “a,” “an,” and “the” include plural referents unless the context clearly dictates otherwise. Thus, for example, reference to “a step” includes reference to one or more of such steps.

As used herein, the term “hydrophilic amino acid” refers to an amino acid selected form the group consisting of: arginine, asparagine, aspartate, glutamine, glutamate, histidine, lysine, serine, and threonine.

As used herein, the term “conserved substitution” refers to an amino acid replacement that changes a given amino acid to a different amino acid with similar biochemical properties (e.g. charge, hydrophobicity and size). For example, an aliphatic amino acid residue is replaced by another aliphatic amino acid residue, while an aromatic amino acid residue is replaced by another aromatic amino acid residue. Table 1 sorts the amino acids into six main classes based on their structure and the general chemical characteristics of their side chains.

TABLE 1

| Class | Amino acids |
|---|---|
| Aliphatic | Glycine, Alanine, Valine, Leucine, |
| | Isoleucine |
| Hydroxyl or sulfur/selenium- | Serine, Cysteine, Selenocysteine, |
| containing | Threonine, Methionine |
| Cyclic | Proline |
| Aromatic | Phenylalanine, Tyrosine, Tryptophan |
| Basic | Histidine, Lysine, Arginine |
| Acidic and their amides | Aspartate, Glutamate, Asparagine, |
| | Glutamine |

Fusion-inhibitory peptides prevent viral entry and have been proven effective against a wide range of viral diseases. Disclosed herein are the lipopeptides that inhibit viral entry into a host cell. Accordingly, also described herein are uses of such peptides for treating a viral infection or inhibiting a viral infection (for example, by inhibiting viral fusion with a host cell).

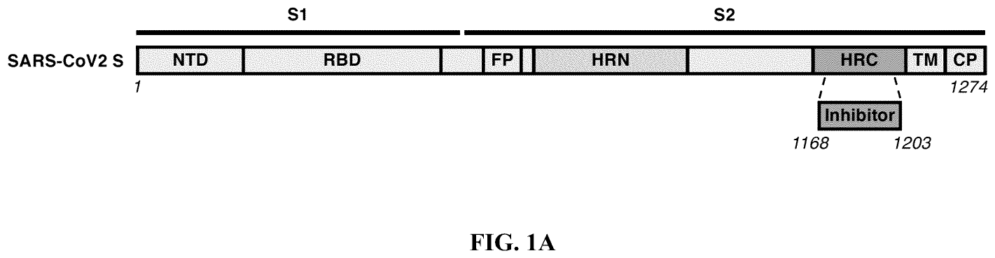

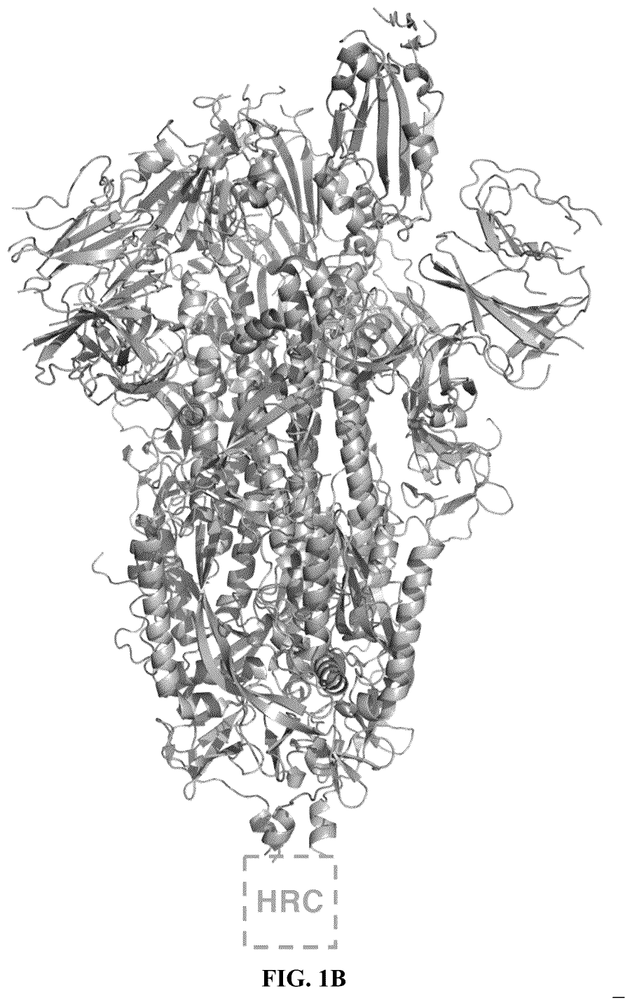

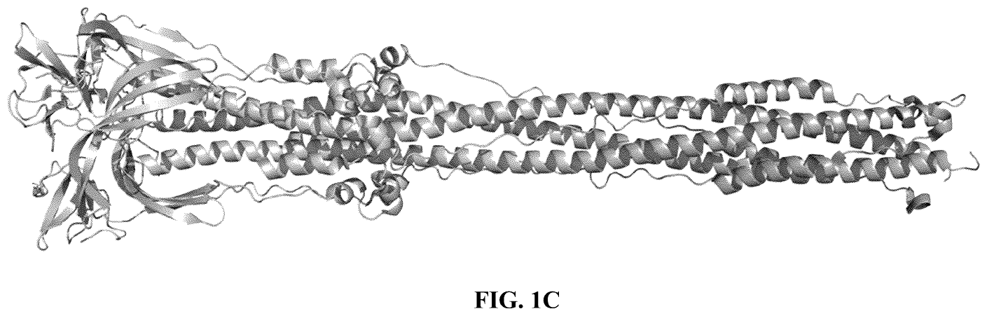

Infection by coronaviruses requires membrane fusion between the viral envelope and the host cell membrane. Depending on the cell type, surface molecule expression, and the coronavirus strain, fusion can occur at either the cell surface or the endosomal membrane. The fusion process is mediated by the viral envelope glycoprotein (S) protein. The S protein is a large homotrimer, each monomer having several domains. Specifically, the S protein is divided into a bulb portion (S1), which is the part that binds to the receptor(s), and a stalk portion (S2), which is responsible for fusion between virion and cell membranes (FIG. 1A). S1 has a receptor-binding domain (RBD), which is distal to the viral membrane and is responsible for cell surface attachment. Membrane merger is mediated by a proximal cell fusion domain (FD). Concerted action by the RBD and FD is required for fusion. Upon viral attachment (and uptake in certain cases), host factors (receptors and proteases) trigger large-scale conformational rearrangements in the FD, driven by formation of an energetically stable six-helix bundle (6HB) that couple protein refolding directly to membrane fusion. The FD is thought to form a transient pre-hairpin intermediate composed of a highly conserved N-terminal heptad repeat (HRN) based trimeric coiled-coil core targeted by the C-terminal heptad repeat-derived fusion inhibitory peptides (referred to as C-peptides). C-peptides inhibit viral fusion and entry in a dominant-negative manner by binding to the pre-hairpin intermediate, thereby preventing formation of the 6HB. For strains that fuse at the cell membrane, standard C-peptides can prevent viral entry, but these peptides are ineffective on strains that fuse in the endosome. The intracellular sequestration of S makes it challenging to develop C-peptide fusion inhibitors against endosomal fusing coronavirus strains.

The receptor-binding domain (RBD) of S1 varies among different coronaviruses. RBDs of HCoV-229E, HCoV-NL63, and HCoV-HKU1 are located in the C-terminal region but not the N-terminal domains of the respective S1 subunits. HCoV has been shown to use either cellular proteins or carbohydrates displayed on the plasma membrane as receptors. Interestingly, all known protein receptors for HCoVs are cell surface peptidase, such as aminopeptidase N (APN) for HCoV-229E, dipeptidyl peptidase 4 (DPP4) for MERS-CoV, and angiotensin converting enzyme 2 (ACE2) for HCoV-NL63, SARS-CoV and SARS-CoV-2. On the other hand, HCoV-OC43 and HCoV-HKU1 employ glycan-based receptors carrying 9-O-acetylated sialic acid.

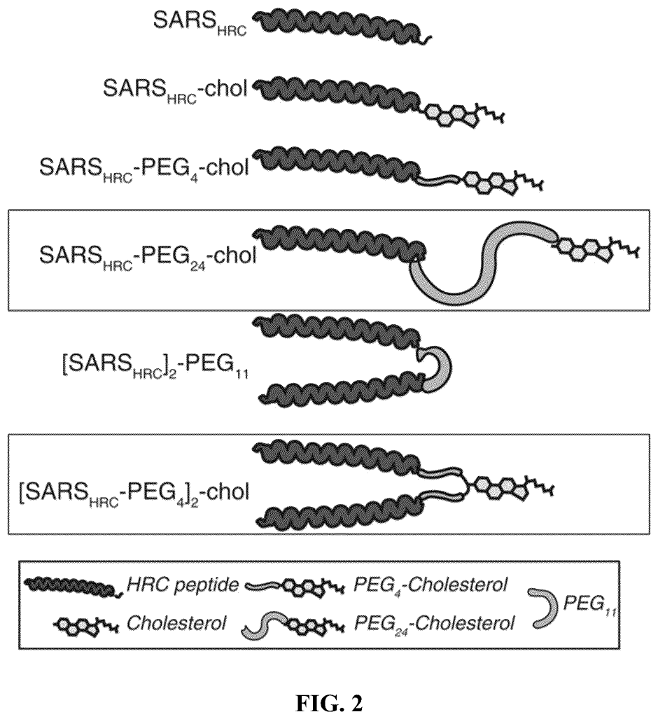

Without being bound by theory, it is believed that the lipopeptides described herein act as an obstruction that rapidly halts the joining or fusion process between the coronavirus and the host cell. The lipopeptides of the disclosure comprise at least one peptide unit that is derived from the HRC of a coronavirus. The lipid portion of the lipopeptide comprises a lipid unit and, optionally, at least one polyethylene glycol linker. In some aspects, the lipid unit is a cholesterol unit. In particular embodiments, the at least one peptide unit is linked to the cholesterol unit via at least one polyethylene glycol linker (FIG. 2). In some aspects, the peptide unit is connected to the polyethylene glycol linker at its C-terminus. Lipopeptides comprising one peptide unit are referred to herein as “monomers”. Lipopeptides comprising two peptide units are referred to herein as a “dimer.” In some aspects, the two peptide units of a dimeric lipopeptide are linked together by a polyethylene glycol linker, for example, which is linked to a cholesterol unit. In other aspects, the two peptide units of a dimeric lipopeptide are linked to one cholesterol unit by separate polyethylene glycol linkers.

### Peptide Unit

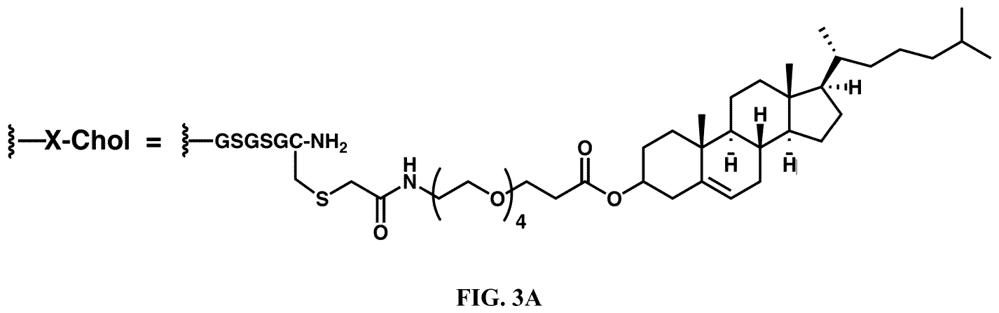

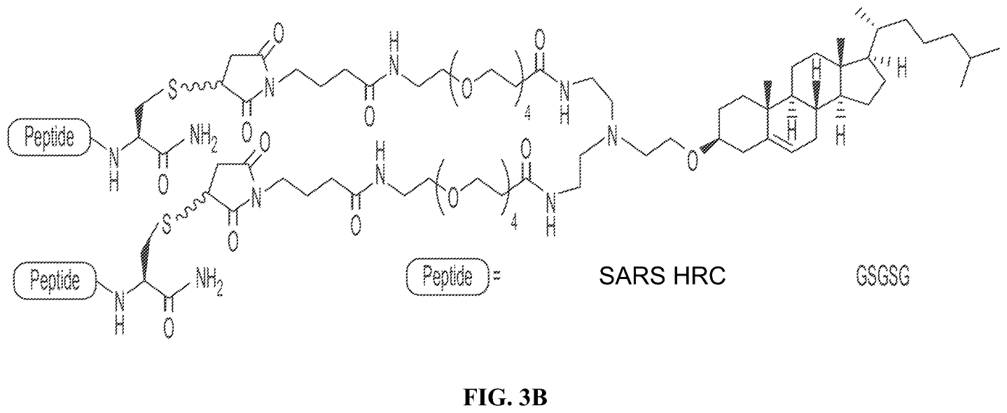

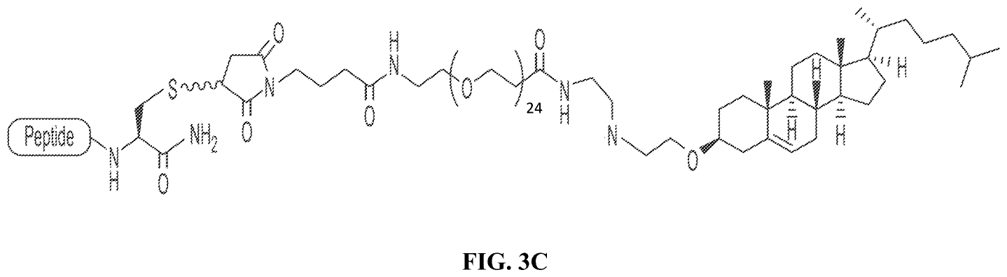

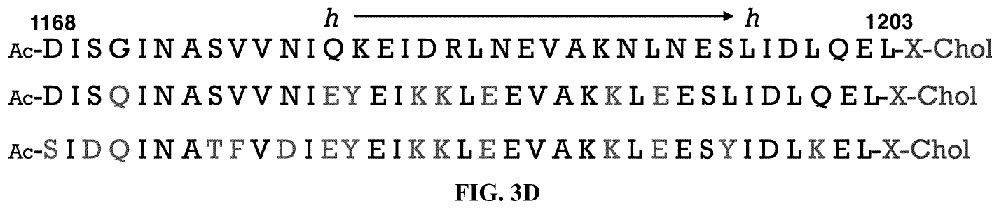

The peptide unit of the lipopeptide has 30-40 amino acid residues in length, wherein the amino acid sequence of the peptide unit has at least 60% sequence identity to an α-helical segment of a C-terminal heptad repeat of a coronavirus S protein while maintaining a secondary structure of an α-helix. In some aspects, the peptide unit comprises 30-40 amino acid residues of the α-helical segment of a C-terminal heptad repeat of a coronavirus S protein, wherein the solvent-exposed residues are substituted with hydrophilic residues (FIG. 3).

In some embodiments, the peptide unit comprises β-amino acid residues, for example, up to 30% of the amino acid residues of the peptide unit are β-amino acid residues. In particular embodiments, between 10% to 30% of the amino acid residues of the peptide unit are β-amino acid residues.

Certain exemplary peptide units are listed in Table 2.

TABLE 2
Exemplary amino acid sequences of the peptide unit. The bolded and
underlined residues are β-amino acid residues.

| | | Amino acid |
|---|---|---|
| SEQ | | sequence derived |
| ID NO. | Amino Acid Sequence | from |
| 1 | DISGINASVVNIQKEIDRLNEVAKNLNESLIDLQEL | SARS-CoV-2 |
| 2 | DISQINASVVNIEYEIKKLEEVAKKLEESLIDLQEL | SARS-CoV-2 |
| 3 | SIDQINATFVDIEYEIKKLEEVAKKLEESYIDLKEL | SARS-CoV-2 |
| 4 | DISQINASVVNIEYEIZKLEUVAZKLEUSLIDLQEL | SARS-CoV-2 |
| 5 | SLDQINVTFLDLEYEMKKLEEAIKKLEESYIDLKEL | HCoV-OC43 |
| 6 | SLTQINTTLLDLTYEMLSLQQVVKALNESYIDLKEL | MERS-CoV |

### Lipid Portion

The lipid portion of the lipopeptide can be any pegylated lipid comprising a polyethylene glycol linker attached to a lipid molecule. For example, the lipid may be selected from the group consisting of: dipalmitoyl phosphatidylcholine (DPPC), distearoyl phosphatidylethanolamine (DSPE), and cholesterol. In some aspects, the lipid portion is a pegylated cholesterol unit. Thus, the peptide unit is linked to cholesterol unit via the polyethylene glycol linker.

The polyethylene glycol linker may be PEG₄, PEG₈, PEG₁₁, PEG₂₄, or PEG₂₈. In some embodiments, the polyethylene glycol linker is PEG₄. In other embodiments, the polyethylene glycol linker is PEG₂₄. Preferably, when the lipopeptide is a dimer, the lipopeptide comprises two PEG₄ linkers.

### Methods of Use

Methods of inhibiting and/or treating an alphacoronavirus infection or embecovirus infection in a subject are described. In some embodiments, the method of inhibiting and/or treating an alphacoronavirus comprises administering to the subject a lipopeptide wherein the peptide unit is derived from the HRC of a betacoronavirus S protein, from example from SARS-CoV-2 or HCoV OC43. In some embodiments, the method of inhibiting and/or treating an embecovirus comprises administering to the subject a lipopeptide wherein the peptide unit is derived from the HRC of SARS-CoV-2 S protein.

In 1965, HCoV 229E was the first of the common human coronaviruses to be described and was followed soon after by serologically distinct OC43. In the following decade it was determined that 15-29% of common colds are attributable to these common human coronaviruses. Four distinct species of human coronaviruses are globally distributed and cause the common cold. HCoVs 229E and NL63 are alphacoronaviruses that cause the common cold and belong in the subgenera Duvinacovirus and Setrcovirus, respectively. HCoVs OC43, and HKU1 are betacoronaviruses that cause the common cold and belong in the subgenus Embecovirus. In the majority of cases, these viruses are self-limiting, but an episode of common cold can cause severe respiratory disease in the elderly and immunocompromised patients. Accordingly, methods of treating a common cold are also described.

### Pharmaceutical Compositions

Uses of the lipopeptide described herein for inhibiting and/or treating an alphacoronavirus infection or embecovirus infection or for treating the common cold are also described. Uses of the lipopeptide described herein for the manufacture of a medicament, for example to inhibit and/or treat an alphacoronavirus infection or embecovirus infection or to treat the common cold are additionally described.

In some aspects, the lipopeptide used for inhibiting and/or treating an alphacoronavirus infection comprises a peptide unit derived from the HRC of a betacoronavirus S protein, from example from SARS-CoV-2 S protein or HCoV OC43 S protein.

In some aspects, the lipopeptide used for inhibiting and/or treating an embecovirus infection or for treating the common cold comprises a peptide unit derived from the HRC of SARS-CoV-2 S protein.

In some implementations, the pharmaceutical composition or the medicament further comprises one or more pharmaceutically acceptable excipients, for example, a filler, a disintegrant, a surfactant, a diluent, a binder, a glidant, and a lubricant and any combination thereof. Depending on the method of administration, the pharmaceutical composition or the medicament may be aerosolized or in the form of a dry powder.

In some aspects, the pharmaceutical composition or the medicament is in a dosage form selected from the group consisting of: a liquid, a paste, a bar, a cake, a powder, a granulate, a chewable, a tablet, a capsule, a lozenge, a fast-melting tablet or wafer, and a sublingual tablet.

### NUMBERED EMBODIMENTS

The present disclosure is exemplified by the numbered embodiments below.

1. A method of preventing a viral infection in a subject exposed or at risk of exposure to a virus other than SARS-CoV-2, comprising administering to a subject in need thereof a lipopeptide comprising at least one peptide unit comprising or consisting of an amino acid sequence having at least 80% identity to SEQ ID NO:1, SEQ ID NO:2, SEQ ID NO:3, or SEQ ID NO:4.

2. A method of inhibiting an interaction between a viral protein other than SARS-CoV-2 spike protein and cellular ACE2 in a subject, comprising administering to a subject in need thereof a lipopeptide comprising at least one peptide unit comprising or consisting of an amino acid sequence having at least 80% identity to SEQ ID NO:1, SEQ ID NO:2, SEQ ID NO:3, or SEQ ID NO:4.

3. The method of embodiment 1 or embodiment 2, wherein the lipopeptide is administered at least once before the subject is exposed to the virus.

4. The method of any one of embodiments 1 to 3, wherein the lipopeptide is administered at least twice before the subject is exposed to the virus.

5. A method of treating a viral disease other than COVID-19 in a subject, comprising administering to a subject in need thereof a lipopeptide comprising at least one peptide unit comprising or consisting of an amino acid sequence having at least 80% identity to SEQ ID NO:1, SEQ ID NO:2, SEQ ID NO:3, or SEQ ID NO:4.

6. A method reducing the severity of a viral infection other than SARS-CoV-2 in a subject, comprising administering to a subject in need thereof a lipopeptide comprising at least one peptide unit comprising or consisting of an amino acid sequence having at least 80% identity to SEQ ID NO:1, SEQ ID NO:2, SEQ ID NO:3, or SEQ ID NO:4.

7. A method of reducing viral load in a subject of a subject infected with or exposed to a virus other than SARS-CoV-2, comprising administering to a subject in need thereof a lipopeptide comprising at least one peptide unit comprising or consisting of an amino acid sequence having at least 80% identity to SEQ ID NO:1, SEQ ID NO:2, SEQ ID NO:3, or SEQ ID NO:4.

8. A method of preventing disease progression in a subject infected by a virus other than SARS-CoV-2, comprising administering to a subject in need thereof a lipopeptide comprising or consisting of at least one peptide unit comprising an amino acid sequence having at least 80% identity to SEQ ID NO:1, SEQ ID NO:2, SEQ ID NO:3, or SEQ ID NO:4.

9. A method of reducing the duration of an infection by a virus other than SARS-CoV-2, comprising administering to a subject in need thereof a lipopeptide comprising at least one peptide unit comprising or consisting of an amino acid sequence having at least 80% identity to SEQ ID NO:1, SEQ ID NO:2, SEQ ID NO:3, or SEQ ID NO:4.

10. A method of reducing the risk of severe disease or death caused by or ancillary to a viral infection in a subject infected with a virus other than SARS-CoV-2, comprising administering to a subject in need thereof a lipopeptide comprising at least one peptide unit comprising or consisting of an amino acid sequence having at least 80% identity to SEQ ID NO:1, SEQ ID NO:2, SEQ ID NO:3, or SEQ ID NO:4.

11. A method of treating a viral infection in a subject infected by a virus other than SARS-CoV-2, comprising administering to a subject in need thereof a lipopeptide comprising at least one peptide unit comprising or consisting of an amino acid sequence having at least 80% identity to SEQ ID NO:1, SEQ ID NO:2, SEQ ID NO:3, or SEQ ID NO:4.

12. The method of any one of embodiments 2 or 5 to 11, wherein the lipopeptide is administered at least once before the subject develops symptoms of viral disease.

13. The method of any one of embodiments 2 or 5 to 12, wherein the lipopeptide is administered at least twice before the subject develops symptoms of viral disease.

14. The method of any one of embodiments 2 or 5 to 13, wherein the lipopeptide is administered at least once after the onset of symptoms of viral disease.

15. The method of any one of embodiments 2 or 5 to 14, wherein the lipopeptide is administered at least twice after the onset of symptoms of viral disease.

16. The method of any one of embodiments 1 to 15, wherein the subject is a mammal, optionally wherein the mammal is a human.

17. The method of any one of embodiments 1 to 16, wherein the virus expresses a protein that utilizes the ACE2 receptor for cellular entry.

18. The method of any one of embodiments 1 to 17, wherein the virus a coronavirus.

19. The method of embodiment 18, wherein the coronavirus is an alphacoronavirus, optionally wherein the alphacoronavirus is HCoV 229E or HCoV NL63.

20. The method of embodiment 18, wherein the coronavirus is an embecovirus, optionally wherein the embecovirus is HCoV OC43 or HCoV HKU1.

21. The method of any one of embodiments 1 to 17, wherein the virus a measles virus.

22. The method of any one of embodiments 1 to 17, wherein the virus is a Nipah virus.

23. The method of any one of embodiments 1 to 17, wherein the virus is an Ebola virus.

24. The method of any one of embodiments 1 to 17, wherein the virus is a parainfluenza virus, optionally wherein the virus is human parainfluenza virus type 3.

25. The method of any one of embodiments 1 to 17, wherein the virus is an influenza virus.

26. The method of any one of embodiments 1 to 25, wherein the at least one peptide unit comprises or consists of an amino acid sequence having at least 85% sequence identity to SEQ ID NO:1, SEQ ID NO:2, SEQ ID NO:3, or SEQ ID NO:4, optionally wherein the % sequence identity is to SEQ ID NO:1.

27. The method of any one of embodiments 1 to 25, wherein the at least one peptide unit comprises or consists of an amino acid sequence having at least 90% sequence identity to SEQ ID NO:1, SEQ ID NO:2, SEQ ID NO:3, or SEQ ID NO:4, optionally wherein the % sequence identity is to SEQ ID NO:1.

28. The method of any one of embodiments 1 to 25, wherein the at least one peptide unit comprises or consists of an amino acid sequence having at least 95% identity to SEQ ID NO:1, SEQ ID NO:2, SEQ ID NO:3, or SEQ ID NO:4, optionally wherein the % sequence identity is to SEQ ID NO:1.

29. The method of any one of embodiments 1 to 25, wherein the at least one peptide unit comprises or consists of an amino acid sequence having at least 97% identity to SEQ ID NO:1, SEQ ID NO:2, SEQ ID NO:3, or SEQ ID NO:4, optionally wherein the % sequence identity is to SEQ ID NO:1.

30. The method of any one of embodiments 1 to 25, wherein the at least one peptide unit comprises or consists of an amino acid sequence having 100% identity to SEQ ID NO:1, SEQ ID NO:2, SEQ ID NO:3, or SEQ ID NO:4, optionally wherein the % sequence identity is to SEQ ID NO:1.

31. The method of any one of embodiments 1 to 30 wherein the at least one peptide unit comprises from 1 to 10 β-amino acids, optionally wherein the at least one peptide unit comprises 1, 2, 3, 4, 5, 6, 7, 8, 9 or 10 β-amino acids.

32. The method of any one of embodiments 1 to 31, wherein the at least one peptide unit further comprises a GSGSGC unit.

33. The method of any one of embodiments 1 to 31, wherein the at least one peptide unit further comprises a GSGSC unit.

34. The method of any one of embodiments 1 to 33, wherein the lipopeptide further comprises at least one lipid unit.

35. The method of embodiment 34, wherein the at least one lipid unit comprises cholesterol, tocopherol, or palmitate.

36. The method of embodiment 34 or embodiment 35, wherein the at least one lipid unit comprises cholesterol.

37. The method of any one of embodiments 1 to 36, wherein the lipopeptide further comprises at least one linker unit.

38. The method of embodiment 37, wherein the at least one linker unit comprises polyethylene glycol (PEG).

39. The method of embodiment 37 or embodiment 38, wherein the at least one linker unit comprises or consists of PEG4, PEG11, PEG24, or PEG28.

40. The method of embodiment 39, wherein, the at least one linker unit comprises or consists of PEG4.

41. The method of any one of embodiments 37 to 40, wherein the at least one linker unit comprises a maleimide group.

42. The method of any one of embodiments 1 to 41, wherein the lipopeptide comprises two peptide units, two linker units, and one lipid unit.

43. The method of embodiment 42, wherein the two peptide units and the two linker units are identical.

44. The method of embodiment 42 or embodiment 43, wherein the lipopeptide has the structure of formula A:

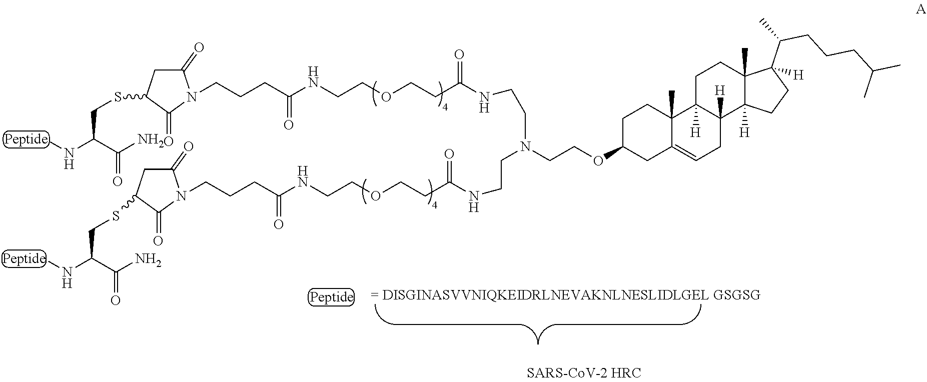

45. The method of any one of embodiments 1 to 41, wherein the lipopeptide comprises one peptide unit, one linker unit, and one lipid unit.

46. The method of any one of embodiments 1 to 44, wherein the lipopeptide is formulated with one or more pharmaceutically acceptable excipients.

47. The method of any one of embodiments 1 to 45, wherein the lipopeptide is formulated in a dosage form selected from the group consisting of: a liquid, a paste, a bar, a cake, a powder, a granulate, a chewable, a tablet, a capsule, a lozenge, a fast-melting tablet or wafer, and a sublingual tablet.

48. The method of any one of embodiments 1 to 20, wherein the lipopeptide is administered subcutaneously.

49. The method of any one of embodiments 1 to 20, wherein the lipopeptide is administered to the subject's airway (e.g., to the mouth, nose, nasal cavity, sinuses, larynx, trachea, and/or one or both bronchial tubes).

50. The method of any one of embodiments 1 to 20, wherein the lipopeptide is administered intranasally.

51. The method of embodiment 50, wherein the lipopeptide is administered as nasal drops or spray.

52. The method of embodiment 50, wherein the lipopeptide is administered as nasal powder.

53. The method of any one of embodiments 1 to 52, wherein the lipopeptide is administered at least once.

54. The method of any one of embodiments 1 to 53, wherein the lipopeptide is administered at least two times.

55. The method of any one of embodiments 1 to 15, wherein the lipopeptide is administered at least once daily.

56. The method of embodiment 55, wherein the lipopeptide is administered twice daily.

57. The method of embodiment 55 or embodiment 56, wherein the lipopeptide is administered for a period of at least one day, at least two days, or at least 3 days.

58. The method of any one of embodiments 1 to 57, wherein the lipopeptide is administered over a treatment period of up to a week.

59. The method of any one of embodiments 1 to 57, wherein the lipopeptide is administered over a treatment period of up to two weeks.

60. A lipopeptide comprising:
(a) at least one peptide unit having 30-40 amino acid residues in length and comprising an amino acid sequence having 100% identity to SEQ ID NO: 1, SEQ ID NO: 2 (D-2), or SEQ ID NO: 3 (D-3);
(b) a cholesterol unit; and
(c) at least one PEG₂₈ polyethylene glycol linker.

61. The lipopeptide of embodiment 60, further comprising a GSGSGC unit or a GSGSC unit.

62. The lipopeptide of embodiment 60 or embodiment 61, wherein the at least one linker comprises a maleimide group.

63. The lipopeptide of any one of embodiments 60 to 62, wherein the lipopeptide comprises one peptide unit, one linker unit, and one lipid unit.

64. The method of any one of embodiments 60 to 63, wherein the lipopeptide comprises two peptide units, two linker units, and one lipid unit.

65. A lipopeptide comprising:
(a) at least one peptide unit having 30-40 amino acid residues in length, wherein:
   (i) the amino acid sequence of the peptide unit has at least 60% sequence identity to an α-helical segment of a C-terminal heptad repeat of a coronavirus S protein,
   (ii) the peptide unit has a secondary structure of α-helix, and
   (iii) 10%-30% of the amino acid residues of the peptide unit are β-amino acid residues;
(b) a cholesterol unit; and
(c) at least one polyethylene glycol linker;
(d) provided:
   (i) the at least one polyethylene glycol linker is PEG₂₈;
   (ii) the at least one peptide unit comprises an amino acid sequence having 100% identity to SEQ ID NO: 4; and/or
   (iii) the lipopeptide comprises two peptide units and two polyethylene glycol linkers, and the two peptide units are each linked to the cholesterol unit through the two polyethylene glycol linkers.

66. The lipopeptide of embodiment 65, wherein the amino acid sequence of the peptide unit has at least 70% sequence identity to the α-helical segment of the C-terminal heptad repeat.

67. The lipopeptide of embodiment 65 or embodiment 66, wherein the amino acid sequence of the peptide unit has at least 80% sequence identity to the α-helical segment of the C-terminal heptad repeat.

68. The lipopeptide of any one of embodiments 65 to 67, wherein the amino acid sequence of the peptide unit has at least 85% sequence identity to the α-helical segment of the C-terminal heptad repeat.

69. The lipopeptide of any one of embodiments 65 to 68, wherein the amino acid sequence of the peptide unit has at least 90% sequence identity to the α-helical segment of the C-terminal heptad repeat.

70. The lipopeptide of any one of embodiments 65 to 69, wherein the amino acid sequence of the peptide unit has at least 95% sequence identity to the α-helical segment of the C-terminal heptad repeat.

71. The lipopeptide of any one of embodiments 65 to 70, wherein the amino acid sequence of the peptide unit has at least 97% sequence identity to the α-helical segment of the C-terminal heptad repeat.

72. The lipopeptide of any one of embodiments 65 to 71, wherein the amino acid sequence of the peptide unit has 100% sequence identity to the α-helical segment of the C-terminal heptad repeat.

73. The lipopeptide of any one of embodiments 65 to 72, wherein the coronavirus S protein is from SARS-CoV-2.

74. The lipopeptide of any one of embodiments 65 to 73, wherein from 4 to 10 amino acid residues of the at least one peptide unit are β-amino acid residues, optionally wherein 4, 5, 6, 7, 8, 9, or 10 amino acid residues of the at least one peptide unit are β-amino acid residues.

75. The lipopeptide of any one of embodiments 65 to 74, wherein the lipopeptide comprises two peptide units which comprise the same amino acid sequence.

76. The lipopeptide of any one of embodiments 65 to 67, wherein the lipopeptide comprises two peptide units which comprise different amino acid sequences.

77. A lipopeptide comprising:
at least one peptide unit having 30-40 amino acid residues in length and comprising an amino acid sequence set forth in SEQ ID NO: 2 (D-2) or SEQ ID NO: 3 (D-3);
a cholesterol unit; and
at least one polyethylene glycol linker.

78. The lipoprotein of embodiment 77, wherein the at least one polyethylene glycol linker is PEG₄, PEG₈, PEG₁₁, PEG₂₄, or PEG₂₈.

79. A lipopeptide comprising:
at least one peptide unit having 30-40 amino acid residues in length, wherein:
the amino acid sequence of the peptide unit has at least 60% sequence identity to an α-helical segment of a C-terminal heptad repeat of a coronavirus S protein, the peptide unit has a secondary structure of α-helix, and 10%-20% of the amino acid residues of the peptide unit are β-amino acid residues;
a cholesterol unit; and
at least one polyethylene glycol linker.

80. The lipopeptide of embodiment 79, wherein the coronavirus S protein is from SARS-CoV-2.

81. The lipoprotein of embodiment 80, wherein at least 4 amino acid residues of the at least one peptide unit are β-amino acid residues.

82. The lipoprotein of embodiment 80, wherein the lipoprotein comprises an amino acid sequence set forth in SEQ ID NO: 2 (D-2) and at least 4 amino acid residues of SEQ ID NO. 2 are β-amino acid residues.

83. The lipoprotein of embodiment 82, wherein the at least one peptide comprises an amino acid sequence set forth in SEQ ID NO: 4 (α/β-F).

84. The lipoprotein of any one of embodiments 77 to 83, wherein the at least one polyethylene glycol linker is PEG₄, PEG₈, PEG₁₁, PEG₂₄, or PEG₂₈.

85. The lipoprotein of any one of embodiments 77 to 84, wherein the lipopeptide comprises two peptide units and two polyethylene glycol linkers, the two peptide units are each linked to the cholesterol unit through the two polyethylene glycol linkers.

86. The lipoprotein of any one of embodiments 77 to 85, wherein the two peptide units comprises the same amino acid sequence.

87. The lipoprotein of any one of embodiments 77 to 86, wherein the two peptide units comprises different amino acid sequences.

88. A method of treating an alphacoronavirus infection in a subject, the method comprising: administering to the subject a lipopeptide comprising:
at least one peptide unit having 30-40 amino acid residues in length, wherein:
the amino acid sequence of the peptide unit has at least 60% sequence identity to an α-helical segment of a C-terminal heptad repeat of a betacoronavirus S protein,
the peptide unit has a secondary structure of α-helix, and
a cholesterol unit; and
at least one polyethylene glycol linker.

89. The method of embodiment 88, wherein the betacoronavirus S protein is from SARS-CoV-2.

90. A method of treating common cold in a subject, the method comprising: administering to the subject a lipopeptide comprising:
at least one peptide unit having 30-40 amino acid residues in length, wherein:
the amino acid sequence of the peptide unit has at least 60% sequence identity to an α-helical segment of a C-terminal heptad repeat of a SARS-CoV-2 S protein,
the peptide unit has a secondary structure of α-helix, and
a cholesterol unit; and
at least one polyethylene glycol linker.

91. A method of treating an embecovirus infection in a subject, the method comprising: administering to the subject a lipopeptide comprising:
at least one peptide unit having 30-40 amino acid residues in length, wherein:
the amino acid sequence of the peptide unit has at least 60% sequence identity to an α-helical segment of a C-terminal heptad repeat of a SARS-CoV-2 S protein,
the peptide unit has a secondary structure of α-helix, and
a cholesterol unit; and
at least one polyethylene glycol linker.

92. The method of any one of embodiments 88 to 91, wherein the subject is administered the lipopeptide within one day of being infected.

93. The method of embodiment 92, wherein the subject is administered the lipopeptide daily until two days after infection.

94. A method of inhibiting an alphacoronavirus infection in a subject, the method comprising: administering to the subject a lipopeptide comprising:
at least one peptide unit having 30-40 amino acid residues in length, wherein:
the amino acid sequence of the peptide unit has at least 60% sequence identity to an α-helical segment of a C-terminal heptad repeat of a betacoronavirus S protein,
the peptide unit has a secondary structure of α-helix, and
a cholesterol unit; and
at least one polyethylene glycol linker.

95. The method of embodiment 94, wherein the alphacoronavirus is a HCoV-229E or HCoV-NL63.

96. The method of embodiment 94 or embodiment 95, wherein the betacoronavirus S protein is from SARS-CoV-2.

97. A method of inhibiting an embecovirus infection in a subject, the method comprising: administering to the subject a lipopeptide comprising:
at least one peptide unit having 30-40 amino acid residues in length, wherein:
the amino acid sequence of the peptide unit has at least 60% sequence identity to an α-helical segment of a C-terminal heptad repeat of a SARS-Cov-2 S protein,
the peptide unit has a secondary structure of α-helix, and
a cholesterol unit; and
at least one polyethylene glycol linker.

98. The method of embodiment 97, wherein the embecovirus is HCoV-HKU1 or HCoV-OC43.

99. The method of any one of embodiments 88 to 98, wherein subject is administered a lipopeptide of any one of embodiments 77 to 87.

100. Use of a lipopeptide derived from betacoronavirus for treating an alphacoronavirus infection, wherein the lipoprotein derived from betacoronavirus comprises:
at least one peptide unit having 30-40 amino acid residues in length, wherein:
the amino acid sequence of the peptide unit has at least 60% sequence identity to an α-helical segment of a C-terminal heptad repeat of a betacoronavirus S protein,
the peptide unit has a secondary structure of α-helix, and
a cholesterol unit; and
at least one polyethylene glycol linker.

101. Use of a lipopeptide derived from betacoronavirus for the manufacture of a medicament for treating an alphacoronavirus infection, wherein the lipoprotein derived from betacoronavirus comprises:
at least one peptide unit having 30-40 amino acid residues in length, wherein:
the amino acid sequence of the peptide unit has at least 60% sequence identity to an α-helical segment of a C-terminal heptad repeat of a betacoronavirus S protein,
the peptide unit has a secondary structure of α-helix, and
a cholesterol unit; and
at least one polyethylene glycol linker.

102. The use of any one of embodiment 100 or embodiment 101, wherein the alphacoronavirus is HCoV-229E or HCoV-NL63.

103. Use of a lipopeptide derived from SARS-CoV-2 for the treatment of an embecovirus infection, wherein the lipoprotein derived from SARS-CoV-2 comprises:
at least one peptide unit having 30-40 amino acid residues in length, wherein:
the amino acid sequence of the peptide unit has at least 60% sequence identity to an α-helical segment of a C-terminal heptad repeat of a SARS-CoV-2 S protein,
the peptide unit has a secondary structure of α-helix, and
a cholesterol unit; and
at least one polyethylene glycol linker.

104. Use of a lipopeptide derived from SARS-CoV-2 for the manufacture of a medicament for treating an embecovirus infection, wherein the lipoprotein derived from SARS-CoV-2 comprises:
at least one peptide unit having 30-40 amino acid residues in length, wherein:
the amino acid sequence of the peptide unit has at least 60% sequence identity to an α-helical segment of a C-terminal heptad repeat of a SARS-CoV-2 S protein,
the peptide unit has a secondary structure of α-helix, and
a cholesterol unit; and
at least one polyethylene glycol linker.

105. The use of embodiment 103 or embodiment 104, wherein the embecovirus is HCoV-HKU1 or HCoV-OC43.

106. Use of a lipopeptide derived from SARS-CoV-2 for treating common cold, wherein the lipoprotein derived from SARS-CoV-2 comprises:
at least one peptide unit having 30-40 amino acid residues in length, wherein:
the amino acid sequence of the peptide unit has at least 60% sequence identity to an α-helical segment of a C-terminal heptad repeat of a SARS-CoV-2 S protein,
the peptide unit has a secondary structure of α-helix, and
a cholesterol unit; and
at least one polyethylene glycol linker.

107. The use of any one of embodiments 100 to 106, wherein the lipopeptide derived from betacoronavirus or from SARS-CoV-2 is the lipopeptide of any one of embodiments 77 to 87.

### EXAMPLES

### Example 1. Lipopeptide Design

After analyzing 5 overlapping peptides and based on sequence alignment with a previously described MERS-CoV HRC-derived fusion inhibitory peptide, a 36-amino acid residue lipopeptide was ultimately chosen to comprise a peptide unit spanning residues 1168-1203 within the HRC domain of the SARS-CoV-2 S protein (FIG. 1). For lipid conjugation, this 36-residue peptide unit was extended at the C-terminus by a Gly-Ser-Gly-Ser-Gly-Cys segment. The cysteine side chain was used as a nucleophilic handle to append a cholesterol unit, with an intervening polyethylene glycol linker, in particular a tetraethylene glycol linker (PEG₄). The cholesterol unit anchors the peptide unit in host cell membranes.

The 42-mer peptide (HRC plus C-terminal extension) was prepared by microwave-assisted solid-phase peptide synthesis protocols, purified by reverse-phase HPLC, conjugated to a bromoacyl tetra-ethylene glycol-cholesterol reagent, as previously described, and purified again by HPLC.

Other cholesterol-conjugated lipopeptides tested include ones with the peptide unit derived from the MERS-CoV HRC domain (corresponding to residues 1251-1286 of MERS-CoV S; SEQ ID NO. 6) and the designed peptide EK1 (SEQ ID NO. 5). These cholesterol-conjugated lipopeptides were prepared using an analogous protocol.

### Example 2. Lipopeptides Effectively Prevent Fusion in Cell Culture

A cell-cell fusion assay based on α-galactosidase (β-gal) complementation was used for initial functional evaluation of the SARS-CoV-2 lipopeptide regarding SARS-CoV-2 S-mediated fusion. For this assay, 293T cells expressing hACE2 and the N-terminal portion of β-gal were mixed with cells expressing the SARS-CoV-2 S protein and the C-terminal portion of β-gal. When fusion mediated by S occurs, the two parts of β-gal combine to generate a catalytically active species, and fusion is detected via the luminescence that results from substrate processing by β-gal. This assay format allows us to test potential inhibitors of SARS-CoV-2 S-mediated membrane fusion.

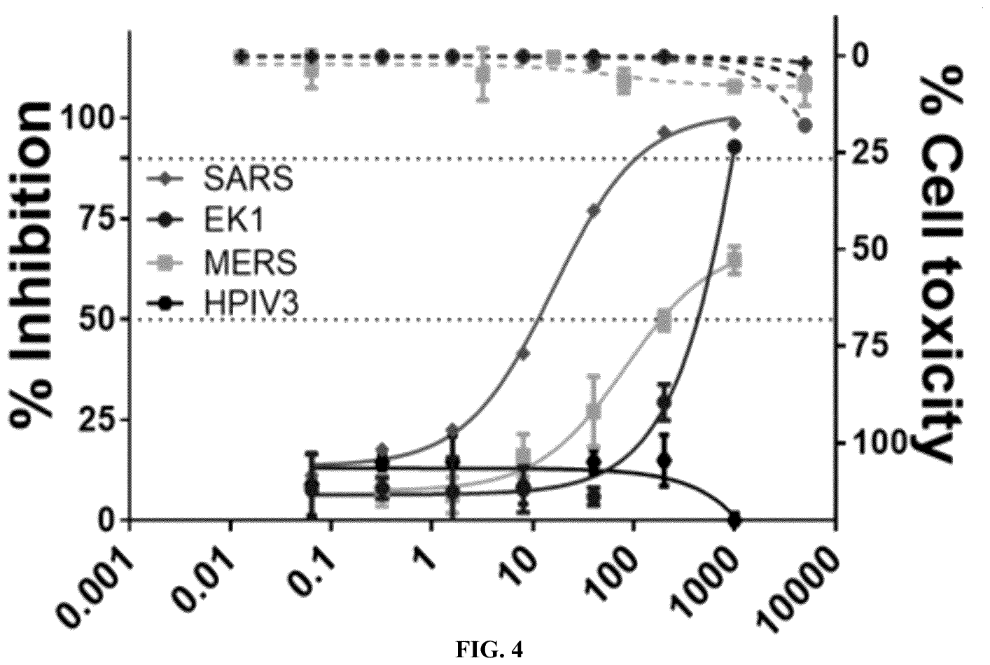

FIG. 4 shows results from this cell-cell fusion assay, where percent inhibition (data shown with solid lines; vertical axis on the left) corresponds to the extent of suppression of the luminescence signal that is observed in the absence of any inhibitor (i.e., 0% inhibition corresponds to maximum luminescence signal). The SARS-CoV-2 C-lipopeptide potently inhibited S-mediated fusion, with IC₅₀˜10 nM and IC₉₀˜100 nM. The lipopeptide based on the human parainfluenza virus type 3 (HPIV3) fusion (F) protein HRC domain, which was used as a negative control, did not inhibit fusion at any concentration tested. The EK1 lipopeptide (EK1C4) inhibited S-mediated fusion in this assay but was substantially less potent (IC₅₀˜300 nM, and IC₉₀>900 nM) than the SARS-CoV-2 lipopeptide. The MERS-CoV C-lipopeptide exhibited only weak inhibition of S-mediated fusion (IC₅₀>650 nM). These data indicate that the SARS-CoV-2 lipopeptide was 30- to 90-fold more effective than the EK1 lipopeptide at inhibiting fusion mediated by SARS-CoV-2 S, based on IC₅₀ and IC₉₀ values, respectively. An MTT assay was performed in parallel during this experiment to evaluate the potential toxicity of each C-lipopeptide (FIG. 4; data shown with dashed lines; vertical axis on the right). Toxicity for each of the C-lipopeptides was minimal (<20%), even at the highest concentrations tested (5 μM). No toxicity was observed for the SARS-CoV-2 C-lipopeptide at its IC₉₀ concentration (100 nM).

Thus, SARS-CoV-2 lipopeptide was more effective than both MERS-CoV and EK-1 lipopeptides at inhibiting fusion mediated by either MERS-CoV or SARS-CoV-2.

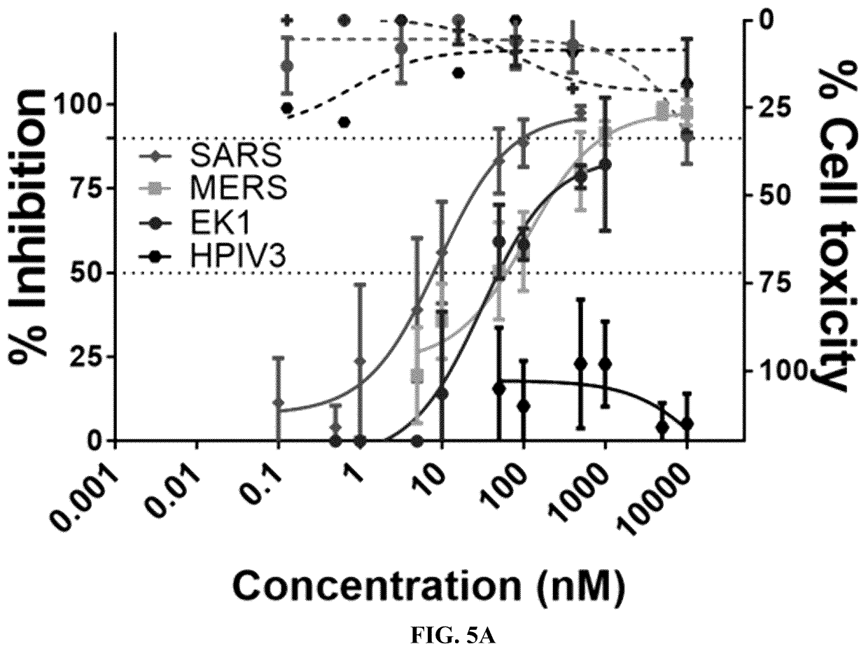

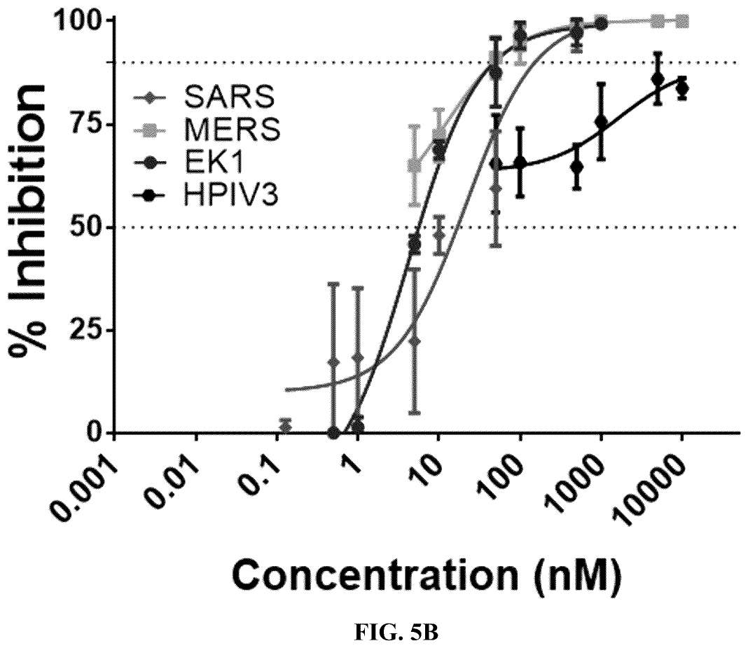

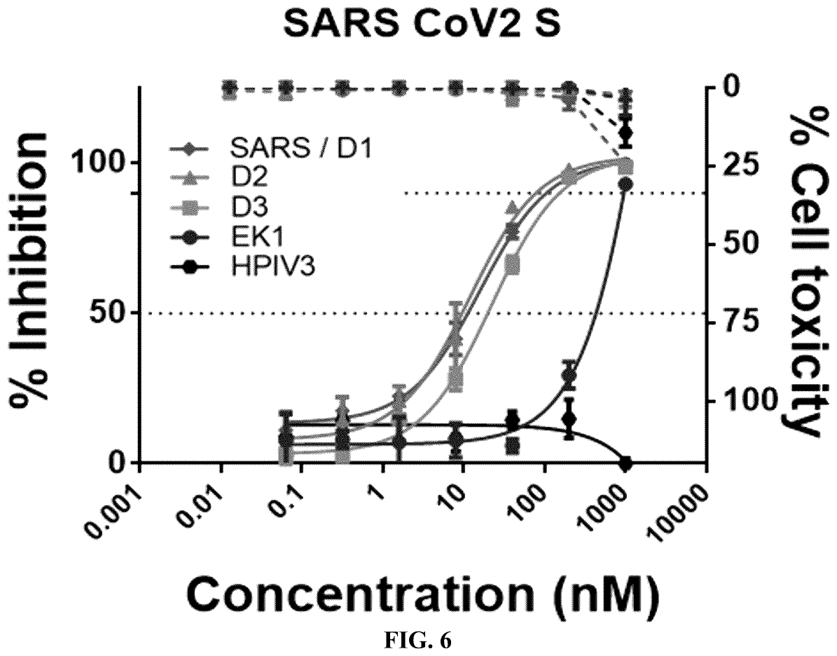

Next, inhibition of SARS-CoV-2 or MERS-CoV infection in Vero E6 cells was studied (FIGS. 5A and 5B, solid lines, left Y-axis). Inhibition of infection was assessed by a plaque-neutralization test. In this assay, the virus was incubated for one hour in the presence of lipopeptide at the concentrations shown and then added to monolayers of cultured Vero E6 cells. After 72 hours, plaques were counted to determine the extent of infection. Similar to results obtained with the fusion assay, the SARS-CoV-2 lipopeptide was a potent inhibitor of SARS-CoV-2 infection (FIG. 5A; IC₅₀˜8 nM). The HPIV3 F HRC lipopeptide was completely inactive against SARS-CoV-2. The EK1 (IC₅₀˜80 nM) and MERS-CoV (IC₅₀˜90 nM) C-lipopeptides were substantially less potent than the SARS-CoV-2 C-lipopeptide against SARS-CoV-2 infection. The SARS-CoV-2 C-lipopeptide was also a potent inhibitor of MERS-CoV infection (FIG. 5B; IC₅₀˜20 nM). Both EK1 (IC₅₀˜8 nM) and MERS-CoV (IC₅₀<8 nM) lipopeptides, however, were slightly more effective than the SARS-CoV-2 lipopeptide at blocking MERS-CoV infection.

Toxicity of C-lipopeptides in Vero E6 cells was evaluated in an MTT assay (FIG. 5A, dotted lines, right Y-axis). The SARS-CoV-2 (toxicity<10%) and EK1 (toxicity<20%) C-lipopeptides demonstrated minimal toxicity at all concentrations less than 1 μM.

### Example 3. SARS-CoV-2 C-Lipopeptide Inhibits SARS-CoV-2 Viral Spread Ex Vivo

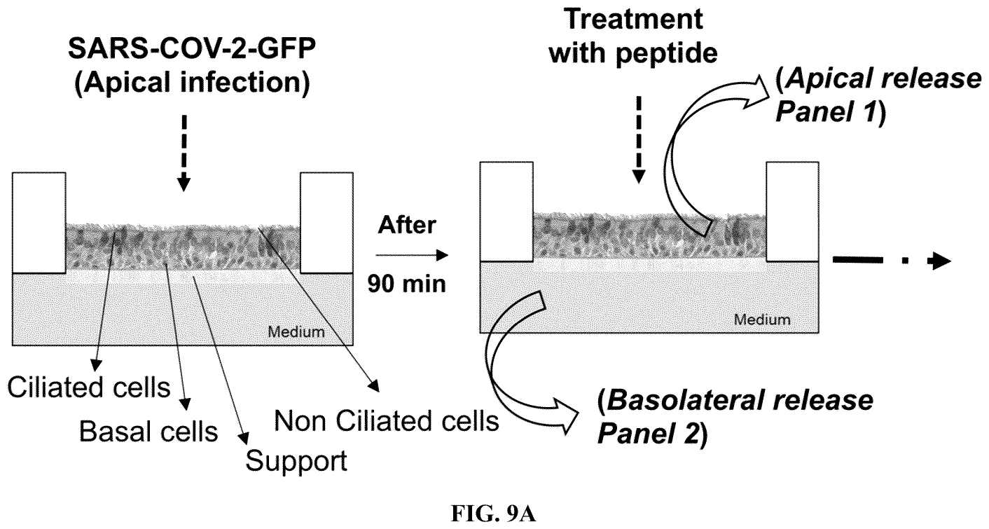

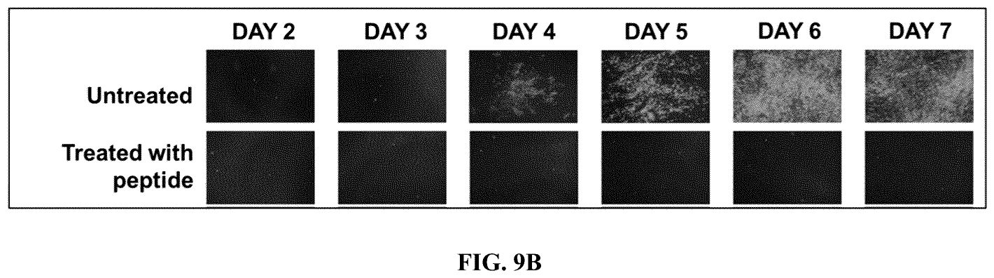

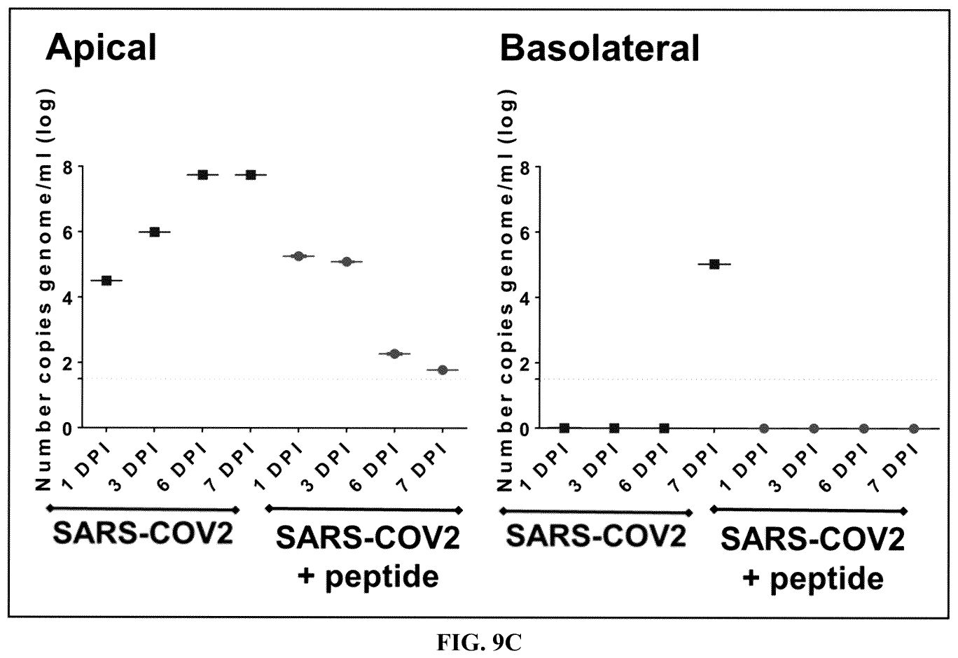

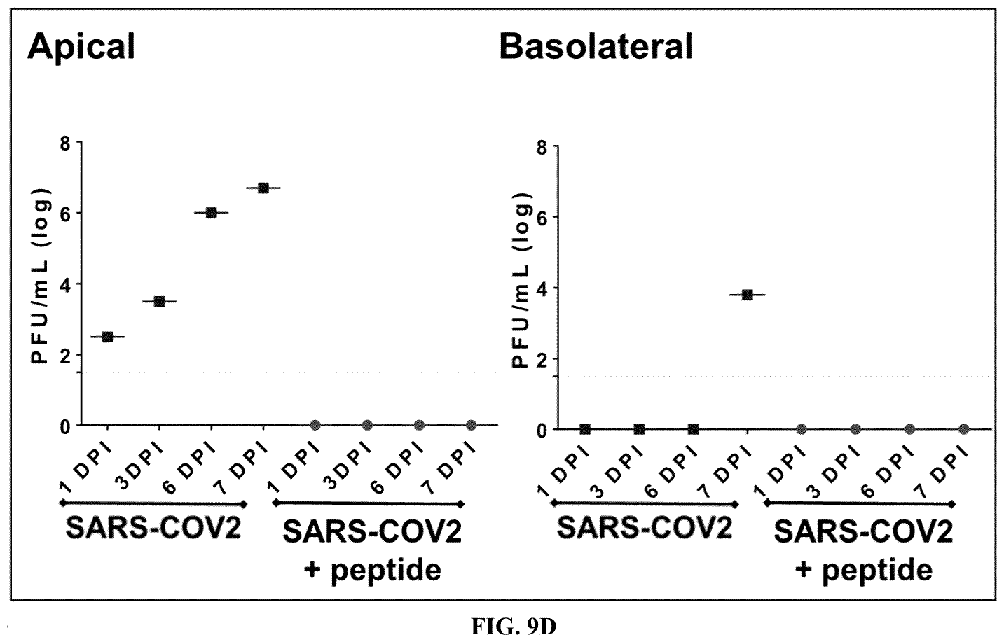

A human airway epithelium (HAE) model was used to study SARS-CoV-2 spread ex vivo. This model (obtained from MatTek, Life Sciences) uses normal, human-derived tracheal/bronchial epithelial (NHBE or TBE) cells that have been cultured at the air-liquid interface to form a polarized pseudo-stratified, highly differentiated tissue that closely resembles the HAE of the respiratory tract (FIG. 9A). HAE has been shown to recapitulate the selective pressures that influence respiratory viral propagation in humans, and HAE studies have been recently used to test small-molecule SARS-CoV-2 inhibitors. It was previously shown that HAE cultures are an ideal ex vivo model to assess fusion-inhibitory peptide activity.

To determine whether the SARS-CoV-2 lipopeptide inhibits the spread of SARS-CoV-2 in human airway tissue, HAE cells were apically inoculated with a SARS-CoV-2 infectious clone expressing a mNeonGreen reporter gene (icSARS-CoV-2-mNG) for 90 minutes, a period that permits viral entry (FIG. 9A). SARS-CoV-2 lipopeptide was directly added (concentration of 200 nM), and subsequently added after 24 and 48 hours. Control wells were mock-treated. Infection was monitored by fluorescence microscopy and apical washes and basolateral medium were collected daily to assess viral spread. Representative fluorescence micrographs are shown in FIG. 9B.

In the absence of lipopeptide treatment, viral spread became evident on day 4, and by day 6, SARS-CoV-2 infection was widespread. In contrast, HAE cultures treated with the SARS-CoV-2 lipopeptide showed no evidence of viral spread during the seven days. Viral infection was quantified by quantitative RT-qPCR and plaque assays to determine the viral genome levels and the amount of infectious virus, respectively (FIGS. 9C and 9D). Data are shown as the average number of RNA genome copies and an average of the plaque-forming units (pfu)/ml in the apical viral collection or basolateral medium from each HAE well in the absence or presence of the SARS-CoV-2 C-lipopeptide. In the untreated wells, viral genome copy number continued to rise, increasing by 4 logs to 10⁸ pfu/ml at day 7. Similarly, increasing levels of infectious virus were detected in these samples. However, in SARS-CoV-2 lipopeptide-treated cultures, viral genome copy numbers declined to almost undetectable levels from day 4 to day 7, demonstrating that the initial infection is extinguished in the airway of peptide-treated cells. No infectious virus was detected from the SARS-CoV-2 lipopeptide-treated wells at any time point. Viral genomes and infectious virus in the basolateral compartment were only detected on day 7 in untreated wells. Viruses from both treated and untreated airway infections at days 3 and 7 were subjected to deep sequencing to identify the potential emergence of resistance mutations. No significant mutations were observed in viruses emerging from the peptide-treated airways. The number of reads in the day 7 treated isolate was nearly as low as in the uninfected negative control, consistent with the low viral load detected by RT-qPCR. Next generation sequencing (NGS) of supernatant fluids, as well as RNAseq of extracted tissue, did not show S specific changes to indicate viral evolution for the HAE model experiment.

### Example 4. In Vivo Biodistribution of Improved C-Lipopeptides

Local and systemic biodistribution of our most potent monomeric and dimeric lipopeptides (SARS_{HRC}-PEG₂₄-chol and [SARS_{HRC}-PEG₄]₂-chol) are compared at 1, 8, and 24 hours after intranasal inoculation or subcutaneous injection in humanized K18 hACE2 mice. The two lipopeptides attained a similar lung concentration at 1 hour after intranasal administration (˜1-2 μM). At 8 and 24 hours, the dimeric [SARS_{HRC}-PEG₄]₂-chol lipopeptide was retained in the lung at high levels with minimal entry into the blood, but the monomeric peptide entered the circulation and led to a decrease in the lung concentration. The dimeric [SARS_{HRC}-PEG₄]₂-chol lipopeptide was distributed throughout the lung after intranasal administration.

### Example 5. Broad-Spectrum Fusion Inhibitor for Pathogenic Coronaviruses

The lipopeptides described herein were observed to inhibit infection of at least 3 out of the 4 human common cold coronaviruses (hCoVs) clinical isolates. Whether [SARS_{HRC}-PEG₄]₂-chol and SARS_{HRC}-PEG₂₄-chol (the two most potent peptides against SARS-CoV-2 infection(1)) inhibited common hCoVs using the human airway epithelia (HAE) was assessed.

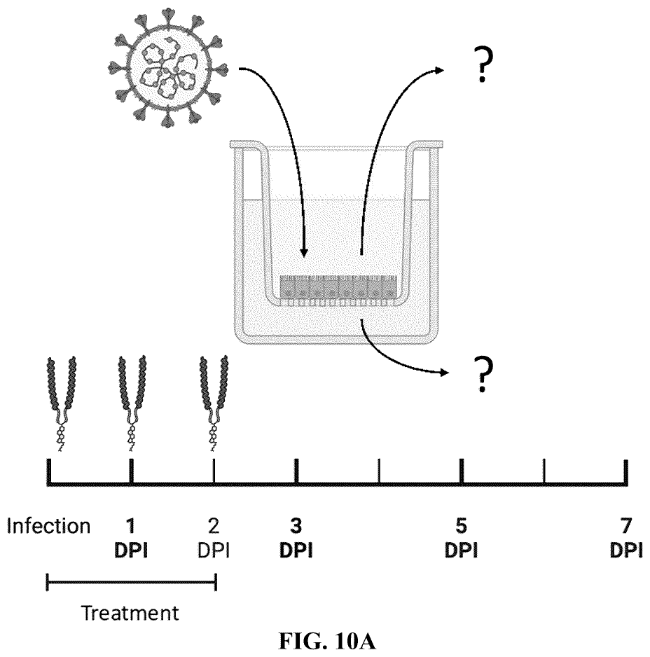

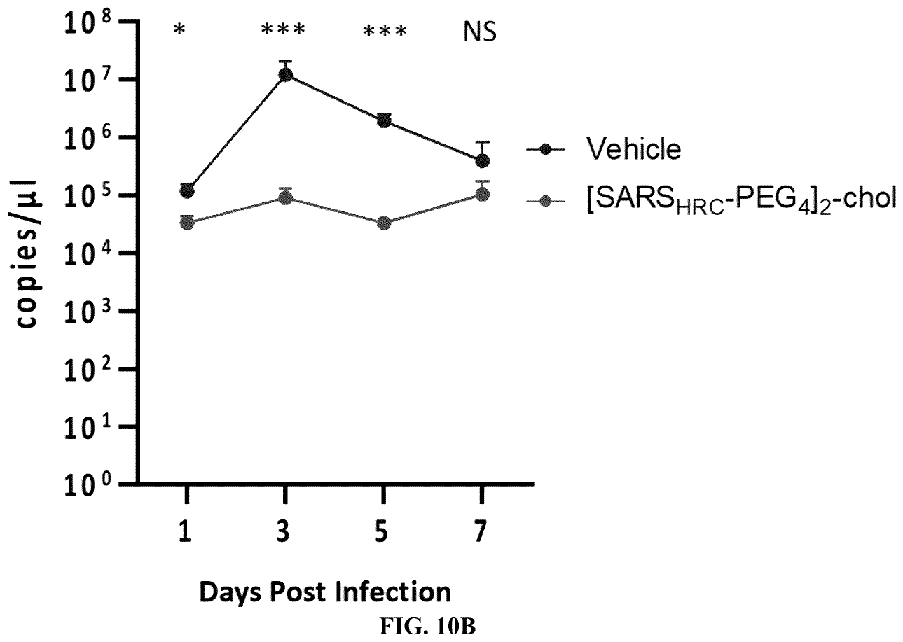

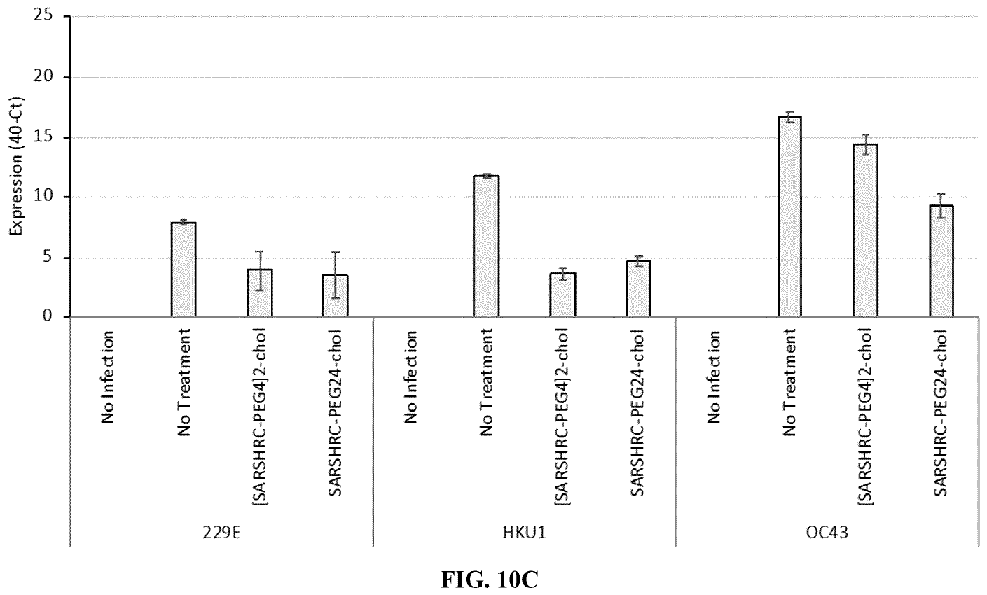

HAE cells were treated with buffer or infected with hCoV-229E, -HKU1, or -OC43 for 90 minutes. Infection occurred in the presence of 1, 5 or 10 μM [SARS_{HRC}-PEG₄]₂-chol or SARS_{HRC}-PEG₂₄-chol on the apical surface of the transwell inserts. After 90 minutes the apical surface was washed and apical washes and basolateral media were collected 1, 3, 5, and 7 days post infection. FIGS. 10B and 10C show the results of the apical washes at day 3 of the well treated with 10 μM of the indicated peptides. The data indicate that both peptides effectively inhibited viral entry when added during the 90-minute infection period.

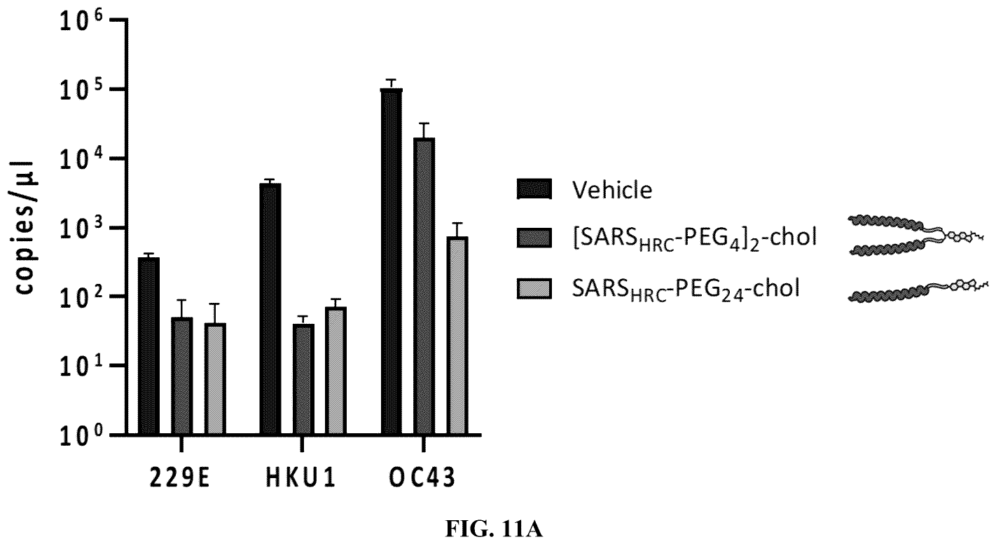

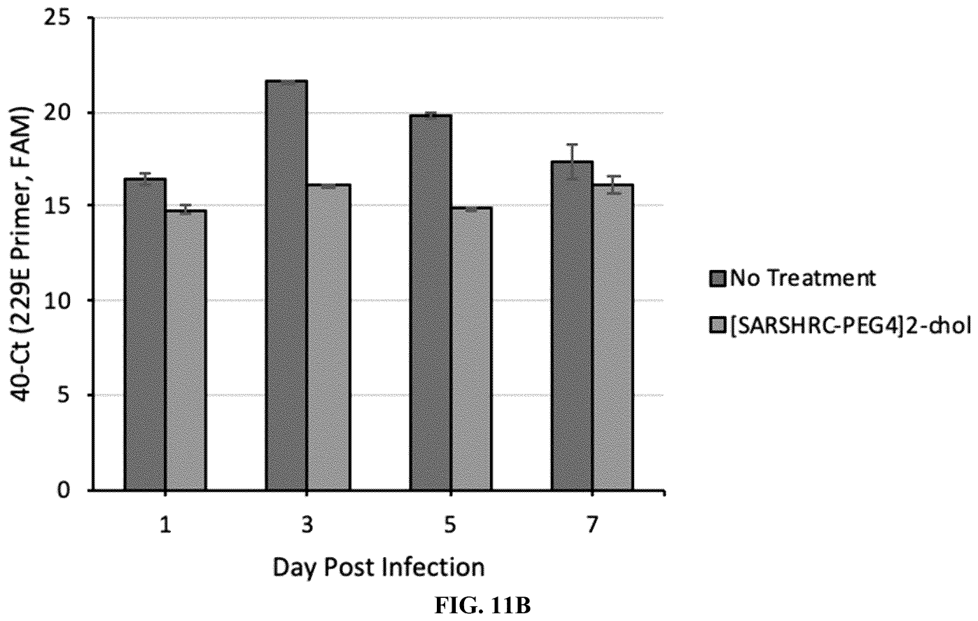

To test the effectiveness after the initial infection, an additional preliminary experiment with 229E was performed. HAE cells were treated with buffer or infected with hCoV-229E for 90 minutes. Immediately following the infection DMSO or 5 μM [SARS_{HRC}-PEG₄]₂-Chol was added to the basolateral media (note that we have published that 7 days of this treatment at 100 μM does not cause any toxicity(1)). New media with DMSO or 5 μM [SARS_{HRC}-PEG₄]₂-Chol was added to the basolateral surface on day 1 and day 2 post infection. Apical washes and basolateral media were collected 1, 3, 5, and 7 days post-infection. FIGS. 11A and 11B show the results of the apical washes. The data indicate that [SARS_{HRC}-PEG₄]₂-Chol effectively inhibited viral spread even after viral entry.

Thus, prophylactic intranasal administration of the lipopeptide prevent infection for all the pathogenic coronaviruses evaluated. The broad-spectrum activity of the SARS-CoV-2-derived lipopeptides supports their use against even newly emergent coronavirus variants.

### Example 6. Lipopeptides Inhibit Direct-Contact Transmission of SARS-CoV-2 in Ferrets

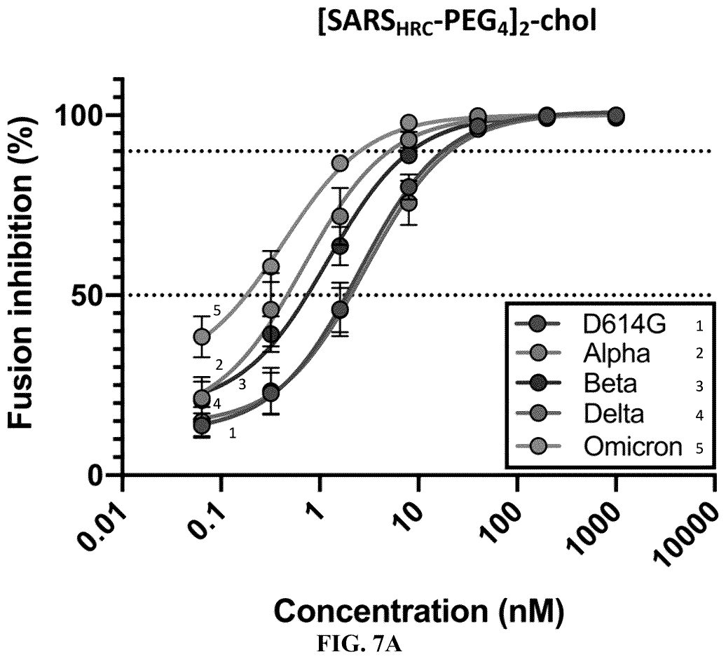

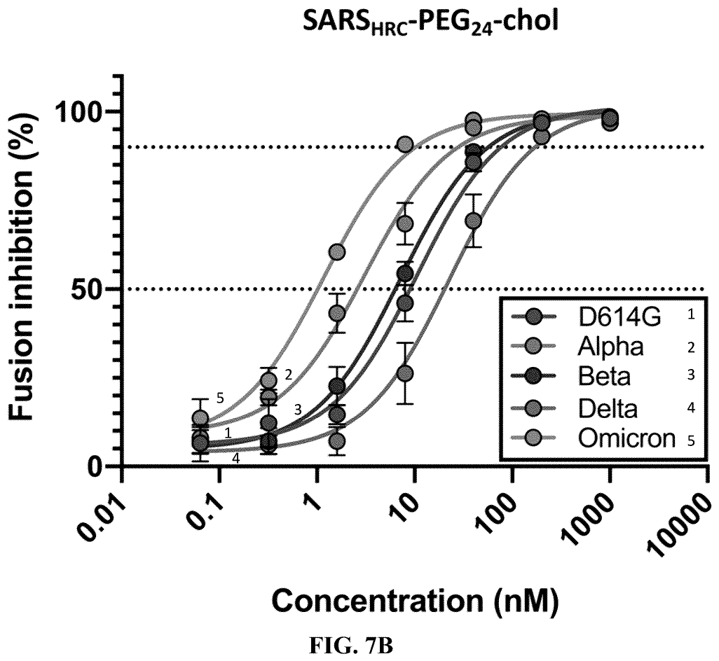

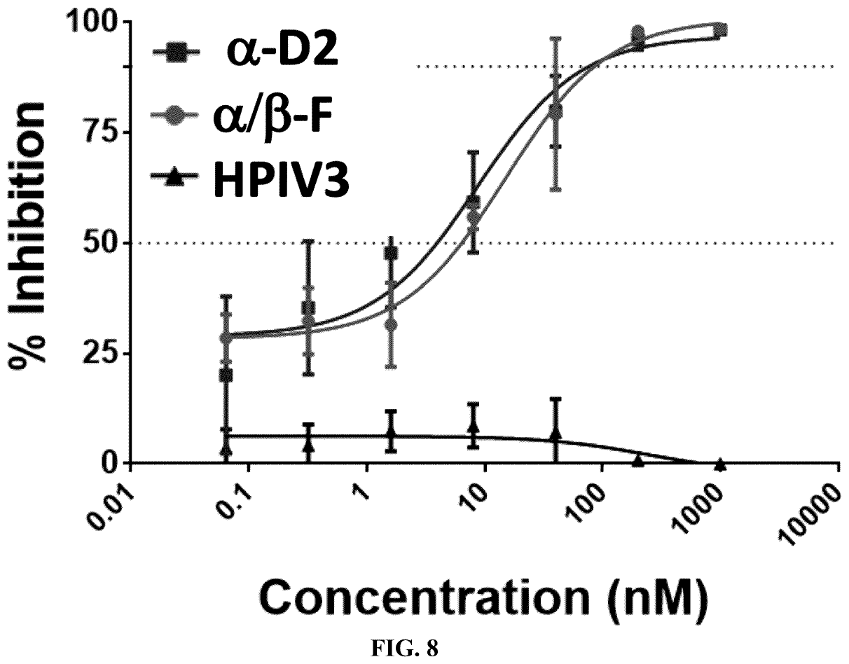

SARS-CoV-2 viral spread ex vivo can be prevented by the described lipopeptide derived from the HRC of SARS-CoV-2 S protein (FIGS. 7A and 7B), while the dimeric form of this lipopeptide is effective in vivo. In a SARS-CoV-2 transmission model in ferrets under high infectious pressure conditions, the transmission inhibited was by the lipopeptide derived from the HRC of SARS-CoV-2 S protein (FIGS. 9A-9D). A dose of 2.7 mg/kg body weight of [SARS_{HRC}-PEG₄]₂-chol administered intranasally once a day prevented direct contact transmission between ferrets.

### Example 7. Ex Vivo Antiviral Activity and Virus Evolution Experiments to Study the Molecular Basis for Antiviral Activity and Resistance to C-Lipopeptide Fusion Inhibitors

To understand the determinants of infection in the natural host, the HAE model described above will be used, which can characterize the polarity and cell specificity of viral infection. A new human developmental lung organoid (LO) model has also been adapted to represents the developing lung as a model for several respiratory viruses. The HAE and LO are ideal for assessing field isolates in experiments that replicate the clinical scenario.

The clinical use of Fuzeon© for HIV-1 resulted in the emergence of drug resistant HIV-1 variants. Escape variant viruses also emerged upon in vitro passaging of HIV-1 in the presence of Fuzeon©. The resistant viral population acquired mutations within a highly conserved stretch of three HRN amino acids, glycine-isoleucine-valine (GIV). Resistance mutations in this GIV motif also exist within the viral quasi-species of patients on Fuzeon© therapy. The resistance was due to either decreased interaction between the viral HRN and Fuzeon©, or increased interaction between viral HRN and HRC. Increased kinetics of fusion led to resistance, but also to viruses whose growth depended on the drug. While anti-coronavirus therapy will be of shorter duration than that for HIV (acute vs. chronic treatment), resistance may be important clinically, as it is for influenza. Based on the results in HIV and influenza, the in vitro data on emergence of resistance will apply directly to in vivo behavior of the viruses under selective pressure of treatment and can be used to predict resistance and preemptively improve C-lipopeptide fusion inhibitor design.

Thus, SARS-CoV and MERS-CoV infections will be performed in the HAE model (commercially acquired) and in the human iPSC derived human lung organoid (LO) model. The same viruses used to generate the data of FIGS. 5A and 5B will be used for the experiments. This virus will be used to monitor viral evolution under lipopeptides' selective pressure in real time.

Serial dilutions of peptide inhibitors will be added either before or after infection to evaluate (i) the effect of the peptides in preventing viral entry; (ii) whether the peptides block viral spread within the tissue after infection.

The HAE and organoid tissue will also be for evidence of toxicity of the peptide using established protocols. Following assessment of antiviral activity in HAE and LO, infections will be performed under the selective pressure of optimized lipopeptide fusion inhibitors to analyze the molecular basis of potential resistance; to predict the possibility of evolution of lipopeptide-resistant viruses; and to provide information that will be used to identify the lipopeptide fusion inhibitors least likely to select for resistance.

The same experiment will also be performed with HCoV infections.

10 lipopeptides will be assessed for ex vivo activity against SARS-CoV, MERS-CoV, and common cold coronaviruses (obtained from de-identified clinical isolates (Cl)). A total of 10-20 CIs will be tested for antiviral efficacy and one for each strain for resistance). Resistance will be evaluated using the most potent 4 lipopeptides.

### 1. Assessment of Ex Vivo Antiviral Activity

Supernatant fluids from HAE and cell suspensions for LO will be divided into three aliquots, and processed for (i) RNAseq at CUIMC and RT-qPCR, (ii) genome sequence analysis (by Alex Greninger), and (iii) viral titer will be assessed at UTMB (as we have done in A3) for the two select agents (SARS-CoV and MERS-CoV).

### 2. Generation of Resistant Variants (for SARS-CoV, MERS-CoV, and Selected Common Cold Coronavirus Clinical Isolates)

To elicit viruses resistant to the inhibitory effect of lipopeptides, HAE and LOs will be infected, and treated with several concentrations of lipopeptides (ranging between 5× and 40× the IC₅₀) for three to four days in HAE (mimicking short treatment) and for up to 30 days in LOs (prolonged treatment). Lipopeptides will be added after the initial infection to allow the viral polymerase complex to replicate and produce phenotypic variants for selection. Resistant viruses would spread even in the presence of C-lipopeptides. Yields of virus will be determined by plaque assays and/or by RT-qPCR (as in FIGS. 9C and 9D). As the virus spreads in the presence of inhibitor, the concentration of the inhibitor will be gradually increased to obtain a population of resistant viruses. Passaged virus will be sequenced as well as tested for inhibitor sensitivity (see FIGS. 5A and 5B). This strategy of applying selective pressure for viral evolution is similar to the informative experiments performed in our lab for neuraminidase-resistant variants⁵⁶ and small molecule inhibitor-resistant variants.

### 3. Analysis of Resistant Variants in Vitro

Mutant resistant viruses, before and after expansion (by growth in HAE), will be sequenced. Resistant virus mutants will be analyzed by high depth, whole viral genome sequencing. Sequences of the HAE-grown viruses will be compared to population-derived sequences generated during the duration of the selection experiments using custom bioinformatics software specifically made for longitudinal analysis of viral evolution. This approach will prevent neglecting potentially important viral sub-populations or alleles present across the genome that may co-exist during or after the selection process. Whether the fitness of each variant is similar to that of the parent virus, or whether the variants require the presence of inhibitor for viability will be determined.

Shotgun sequencing enables a simple, one-workflow protocol for all RNA viruses, while tiling RT-PCR enables specific selection of viral sequences from complex sample types. We will sequence these viruses to a minimum average depth of 200× and call all variants with an allele frequency>4%. Sequence reads will be analyzed using a custom bioinformatic pipeline for longitudinal analysis of viral alleles in which reads for each sample are aligned to a de novo assembly consensus reference of the day/passage 0 viral genome and changes in viral allele frequency are plotted by location and time in an interactive HTML file that allows the user to toggle alleles by depth and allele frequency simultaneously (Iketani et al. “Viral Entry Properties Required for Fitness in Humans Are Lost Through Rapid Genomic Change during Viral Isolation.” mBio 2018, 9(4). DOI: 10.1128/mBio.00898-18). If S contains mutations, the mutated genes will be introduced into expression vectors, and the glycoprotein functions will be evaluated in functional assays. If multiple mutations are found, site-specific mutagenesis will be used to introduce the mutations into the S background, and singly mutated genes will be analyzed for their phenotypes using the same in vitro assays. Location and conservation of the mutations will tell us the extent to which the resistance mechanism(s) for different peptides are similar. If the mutants derived from different peptides are markedly different, we will analyze the contributions of the specific mutations to dissect each contribution.

### 4. Analysis of Resistant Variants in Vivo:

If resistant variants that grow well in vitro and ex vivo are identified, their in vivo fitness will be assessed. Resistant variants' pathogenicity will be compared in vivo to the parent virus (only for SARS-CoV and MERS-CoV). The total number of animals will depend on the number of resistant variants. The mouse models to assess the resistant variants and the peptides' efficacy is human angiotensin-converting enzyme 2 (ACE2) transgenic mouse (hACE2 mouse) for SARS-CoV and in human dipeptidyl peptidase-4 (DPP4) transgenic mice (hDDP4). These models has been shown to be a lethal model for SAR-CoV and MERS-CoV. Both gross pathology and histopathology can be easily observed at both day 3 and day 5 post infection. Viral titers of 10⁶-107 pfu/ml were obtained after 1-3 days post infection.

### 5. Sample Collection and Analysis:

Tissue samples of all major organs will be collected from mice for histopathology and viral load (by RT-qPCR). Virus isolation will be done only from specimens positive for RT-qPCR. Virus titration will be performed by plaque assay. Samples will also be sequenced to assess viral evolution in vivo.

### Example 8. Evaluate the Protection Afforded by Novel Lipopeptide Fusion Inhibitors Against Infection by SARS-CoV and MERS-CoV in Humanized Mice

Pharmacokinetics and efficacy studies will be conducted in mice using the existing lipopeptide lead inhibitor ([SARS_{HRC}-PEG₄]₂-chol) as comparison against its variants and lipopeptides derived from other betacoronaviruses. Whether the in vitro improved lipopeptides identified Example 5 have the desired half-life and tissue biodistribution profiles, and whether they are safe and well tolerated in vivo will be evaluated. The hACE2 and hDDP4 mouse models described above will be used to assess the in vivo anti-coronavirus (both SARS-CoV and MERS-CoV) potency of optimized C-lipopeptide inhibitors. In an iterative process, the outcome of these experiments will guide further optimization so as to provide a set of promising investigational anti-coronavirus agents.

The mice will be administered the lipopeptides subcutaneously (s.q.), intraperitoneally (i.p.), intranasally (i.n.), or intratracheally (i.t.) at a dose of 5 mg/kg). i.t. administration provides more consistent results compared to intranasal delivery, and this will be key for the biodistribution study in as well as for the prophylactic work. Animals will be inoculated with fusion inhibitory C-lipopeptides and sacrificed 12, 24, 36 and 48 hrs later. Serum and organs (lung, liver, spleen, and brain) will be collected. The organs will be split and either frozen with cold isopentane on dry ice for immunofluorescence or homogenized for semi-quantitative ELISA analysis.

### 1. Peptide Dosage

The current prototype [SARS_{HRC}-PEG₄]₂-chol can be dissolved to 40 mg/ml in aqueous buffer. The dosage of this peptide—intranasally administered—can easily reach 50 mg/kg (25 μl=1 mg in a ˜20 g mouse). For the new C-lipopeptides, their solubility, potency, and half-life will be determined.

### 2. Immunofluorescence

The cryo-sections will be stained with specific rabbit anti-HRC antibody (already in hand for both the α- and the α/β-peptides). Tissue sections will be analyzed using fluorescent microscopy.

### 3. ELISA for Biodistribution Studies

Organs will be homogenized using a “BeadBug” homogenizer. Peptide concentration in tissue samples and serum will be determined. Standard curves will be established for the lead peptides, using the same ELISA conditions as for the test samples using antibodies against the aa sequence.

These experiments will determine the effective half-life for the therapeutic dose and the biodistribution of C-lipopeptide inhibitors in mice. There is a possibility that a combination of the different delivery routes (s.q., i.p., i.n., and i.t.) may yield a favorable balance between biodistribution profile and ease of use with minimal adverse side-effects. Delivery to mucosal surfaces (e.g., i.n. and/or lung delivery) would be an easy and effective way to treat prophylactically, and this strategy would be applicable in the field or in hospitals (e.g., to protect health care providers), however for ill patients in intensive care units or others who cannot tolerate i.n. medication, parenteral administration will be preferable. It is expected that i.n. administration with a large volume (i.e., 50 μl) will result in consistent lung delivery, this will be assessed by comparison to i.t., since i.t. has been shown to best mimic lung delivery. This will be important for the in vivo challenge since (especially for prophylaxis) all animals should receive consistent dosage via i.n. In case i.n. does not consistently provide distribution similar to i.t, i.t. may be an alternative route for the experiments that require a single prophylactic dose. The peptides modified with the β-amino acids are anticipated to have longer half-lives.

### Example 9. Evaluation of C-Lipopeptide Toxicity in Mice

Potential side effects and the kinetics of drug clearance will be assessed for the lipopeptides. It is encouraging to note that, in vivo paramyxovirus experiments and in preliminary pharmacokinetic studies, no toxic effects were observed in mice and hamsters treated for up to 21 days at 20 mg/kg.

Acute systemic toxicity testing will be performed in mice to evaluate the toxicity, dose tolerance of the improved lipopeptide analogs. Briefly, purpose-bred outbred mice (n=6 per group, 3 male and 3 female) will receive a single s.q. injection of 20, 50 or 200 mg/kg of fusion inhibitory lipopeptide. Harlan isovolumetric saline will serve as the control. Animals will be closely monitored for survival and/or signs of distress. For acute toxicity: 2 peptides×6 mice×1 inoculation routes×2 doses=24 mice

Additional studies will examine the 15-day toxicity of C-lipopeptides in mice. The animals will be inoculated s.q. with peptide (20 mg/kg) for 15 consecutive days, and monitored daily for weight gain and changes in behavior or appearance (i.e., feeding, ruffled fur, mobility, etc.). Isovolumetric saline will serve as the control in the experiment. For 15 days toxicity: 2 peptides×6 mice×1 inoculation routes=12 mice.

To examine chronic toxicity, C-lipopeptide inhibitors will be administered s.q. and i.n. to mice (n=6 per group) twice weekly at a dose of 20 mg/kg for 30 (i.e., 8 inoculations) or 60 (i.e., 16 inoculations) days. On days 30 and 60, animals will be sacrificed for determination of body and organ weights and gross pathologic examination. Histopathologic sections of brain, liver, lung, spleen and kidney will be also taken on days 30 and 60. Fixed tissues will be embedded in paraffin, stained with hematoxylin and eosin, and examined under a light microscope. Statistical significance of the mean of the treated group compared with that of the control group will be analyzed by a one-way analysis of variance, followed by Dunnett's multiple comparison tests using the Prism program (Graphpad, San Diego). Differences will be considered statistically significant if p<0.05.

For chronic toxicity studies: 2 peptides×6 mice×1 inoculation routes×2 doses=12 mice.

### Example 10. C-Lipopeptide Immunogenicity Studies

Measurement of antibodies associated with administration of optimized peptides will be performed when conducting repeated dose toxicity studies. Effects of antibody responses on pharmacokinetics, incidence and/or severity of adverse effects, complement activation, or pathological changes related to immune complex formation and deposition will be evaluated.

These studies will determine whether the optimized C-lipopeptide fusion inhibitor is non-toxic at likely therapeutic doses and thus determine its therapeutic potential.

### Example 11. In Vivo Efficacy of Improved C-Lipopeptides

The proposed challenge experiments will determine the minimum required dose for prophylactic efficacy and the therapeutic time window for lipopeptide inhibitor efficacy for SARS-CoV and MERS-CoV. First, whether protection is afforded by i.n. peptide administration, prior to, concomitant with, or up to 4 days after the infection will be determined. The optimal dosage will be assessed. Prophylactic and therapeutic studies using alternative delivery routes (s.q.) will also be performed.

### 1. Dosing

For the initial screening of optimized lipopeptide inhibitors, and for determining the optimal dose, groups of 10 animals will be treated with 3 different doses (i.n. and s.q.) one day prior, the day of challenge and then daily (for 2 days). Infection will be performed with 10⁴TCID50 of SARS-CoV (this is more than 50 times the 50% lethal dose in this model since 230 pfu of SARS-CoV are already 100% lethal) and 105 pfu of MERS-CoV i.n. Animals will be weighed daily and euthanized at 5 days post-infection, or in case of distress and/or >20% weight loss as determined by a clinical scoring sheet. The dosage range will be chosen based on the PK and in vitro efficacy data. The peptides will be delivered in aqueous buffer as in our previous work. Control groups will include untreated and vehicle-treated animals as well as those treated with scrambled peptide controls.

For dosing: (2 peptides+scrambled+mock treated)×10 mice×2 routes×3 doses×2 viruses=480 mice.

### 2. Efficacy

Once an effective dose is determined, the therapeutic window for post-exposure treatment will need to be determined. This is particularly important, as this is an important likely use of the product to manage an outbreak. How many days after infection a peptide treatment can provide protection will be determined. Treatments (i.n.) of animals will be initiated on day 1, 2, or 3 days post-infection. Clinical and body weight assessments will be performed as described (at UTMB). At day 7 post infection animals will be euthanized and their lungs, spleen, liver and serum collected for analysis of virus replication and for histopathologic and immunohistochemical analysis.

For efficacy: 2 peptide×10 mice×1 inoculation route×3 time points×2 viruses=120+20 untreated mice

### 3. Viral Load

From lung will be determined by plaque assay on Vero cells and RT-qPCR. Sample tissues from treated and untreated animals will also be sent for sequencing to determine whether viral evolution occurred during treatment.

### 4. Immune Response and Neutralization Assays

Surviving mice will be monitored for humoral immune responses to viral antigens (i.e., neutralization properties of sera from surviving animals will be assessed).

### 5. Histopathology

Tissue samples of lung, spleen and liver will be collected from each mouse for histopathologic and immunohistochemical analysis. Samples will be immersion-fixed in 10% neutral buffered formalin. Replicate sections of lung, spleen and liver will be stained with hematoxylin to detect major histological alterations.

The prophylactic and therapeutic efficacy of two fusion-inhibitory peptides in mouse models of infection will be defined. The readout of the models is clear, and statistically significant groups will be formed. Animals of both sexes will be used to ensure the capture of sex as a variable. The prophylactic administration of the peptides is expected protect against infection. The effect of peptide treatment on the course of infection will be determined; e.g., whether early C-lipopeptide treatment permits the development of an antiviral immune response, and whether the course of disease and organ damage is different in treated and untreated animals. Whether the peptides are also effective in a post-exposure regimen will be determined. Intranasal delivery will likely work well for prophylaxis but it is possible that s.q. will work better after initial infection.

### REFERENCES

Agu R U, Valiveti S, Earles D C, et al. Intranasal delivery of recombinant human parathyroid hormone [hPTH (1-34)], teriparatide in rats. Endocr Res 2004; 30(3):455-67. DOI: 10.1081/erc-200035957.
Augustin J M, Kuzina V, Andersen S B, Bak S. Molecular activities, biosynthesis and evolution of triterpenoid saponins. Phytochemistry 2011; 72(6):435-57. DOI: 10.1016/j.phytochem.2011.01.015.
Augusto M T, Hollmann A, Castanho M A, Porotto M, Pessi A, Santos N C. Improvement of HIV fusion inhibitor C34 efficacy by membrane anchoring and enhanced exposure. J Antimicrob Chemother 2014; 69(5):1286-97.
Bosch B J, van der Zee R, de Haan C A, Rottier P J. The coronavirus spike protein is a class I virus fusion protein: structural and functional characterization of the fusion core complex. J Virol 2003; 77(16):8801-11. DOI: 10.1128/jvi.77.16.8801-8811.2003.
Chen Y W, Huang S X, de Carvalho A, et al. A three-dimensional model of human lung development and disease from pluripotent stem cells. Nat Cell Biol 2017; 19(5):542-549. DOI: 10.1038/ncb3510.
de Vries R D, Schmitz K S, Bovier F T, et al. Intranasal fusion inhibitory lipopeptide prevents direct-contact SARS-CoV-2 transmission in ferrets. Science 2021; 371(6536):1379-1382. DOI: 10.1126/science.abf4896.
Ding N, Chen Q, Zhang W, Ren S, Guo Y, Li Y. Structure-activity relationships of saponin derivatives: a series of entry inhibitors for highly pathogenic H5N1 influenza virus. European journal of medicinal chemistry 2012; 53:316-26. DOI: 10.1016/j.ejmech.2012.04.022.
Figueira T N, Augusto M T, Rybkina K, et al. Effective in Vivo Targeting of Influenza Virus through a Cell-Penetrating/Fusion Inhibitor Tandem Peptide Anchored to the Plasma Membrane. Bioconjug Chem 2018; 29(10):3362-3376. DOI: 10.1021/acs.bioconjchem.8b00527.
Figueira T N, Freire J M, Cunha-Santos C, et al. Quantitative analysis of molecular partition towards lipid membranes using surface plasmon resonance. Sci Rep 2017; 7:45647. DOI: 10.1038/srep45647.
Figueira T N, Mendonca D A, Gaspar D, et al. Structure-Stability-Function Mechanistic Links in the Anti-Measles Virus Action of Tocopherol-Derivatized Peptide Nanoparticles. ACS Nano 2018; 12(10):9855-9865. DOI: 10.1021/acsnano.8b01422.
Figueira T N, Palermo L M, Veiga A S, et al. In Vivo Efficacy of Measles Virus Fusion Protein-Derived Peptides Is Modulated by the Properties of Self-Assembly and Membrane Residence. J Virol 2017; 91(1). DOI: 10.1128/JVI.01554-16.
Gomes B, Santos N C, Porotto M. Biophysical Properties and Antiviral Activities of Measles Fusion Protein Derived Peptide Conjugated with 25-Hydroxycholesterol. Molecules 2017; 22(11). DOI: 10.3390/molecules22111869.
Goya S, Valinotto L E, Tittarelli E, et al. An optimized methodology for whole genome sequencing of RNA respiratory viruses from nasopharyngeal aspirates. PLoS One 2018; 13(6):e0199714. DOI: 10.1371/journal.pone.0199714.
Gralinski L E, Baric R S. Molecular pathology of emerging coronavirus infections. J Pathol 2015; 235(2):185-95. DOI: 10.1002/path.4454.
Greninger A L, Zerr D M, Qin X, et al. Rapid Metagenomic Next-Generation Sequencing during an Investigation of Hospital-Acquired Human Parainfluenza Virus 3 Infections. J Clin Microbiol 2017; 55(1):177-182. DOI: 10.1128/JCM.01881-16.
Hernandez L D, Hoffman L R, Wolfsberg T G, White J M. Virus-cell and cell-cell fusion. Annual review of cell and developmental biology 1996; 12:627-61. DOI: 10.1146/annurev.cellbio.12.1.627.
Hoffmann M, Kleine-Weber H, Schroeder S, et al. SARS-CoV-2 Cell Entry Depends on ACE2 and TMPRSS2 and Is Blocked by a Clinically Proven Protease Inhibitor. Cell 2020; 181(2):271-280 e8. DOI: 10.1016/j.cell.2020.02.052.
Iketani S, Shean R C, Ferren M, et al. Viral Entry Properties Required for Fitness in Humans Are Lost through Rapid Genomic Change during Viral Isolation. mBio 2018; 9(4). DOI: 10.1128/mBio.00898-18.
Ingallinella P, Bianchi E, Ladwa N A, et al. Addition of a cholesterol group to an HIV-1 peptide fusion inhibitor dramatically increases its antiviral potency. Proc Natl Acad Sci USA 2009; 106(14):5801-6.
Kleine-Weber H, Schroeder S, Kruger N, et al. Polymorphisms in dipeptidyl peptidase 4 reduce host cell entry of Middle East respiratory syndrome coronavirus. Emerg Microbes Infect 2020; 9(1):155-168. DOI: 10.1080/22221751.2020.1713705.
Komabayashi K, Matoba Y, Seto J, et al. Isolation of human coronaviruses OC43, HKU1, NL63, and 229E in Yamagata, Japan, using primary human airway epithelium cells cultured by employing an air-liquid interface culture. Jpn J Infect Dis 2020. DOI: 10.7883/yoken.JJID.2020.776.
Kuzina V, Nielsen J K, Augustin J M, Torp A M, Bak S, Andersen S B. Barbarea vulgaris linkage map and quantitative trait loci for saponins, glucosinolates, hairiness and resistance to the herbivore Phyllotreta nemorum. Phytochemistry 2011; 72(2-3):188-98. DOI: 10.1016/j.phytochem.2010.11.007.
Lee K K, Pessi A, Gui L, et al. Capturing a fusion intermediate of influenza hemagglutinin with a cholesterol-conjugated peptide, a new antiviral strategy for influenza virus. The Journal of biological chemistry 2011; 286(49):42141-9. DOI: 10.1074/jbc.M111.254243.
Letko M, Marzi A, Munster V. Functional assessment of cell entry and receptor usage for SARS-CoV-2 and other lineage B betacoronaviruses. Nat Microbiol 2020; 5(4):562-569. DOI: 10.1038/s41564-020-0688-y.
Li F, Liu X, Tang M, et al. Structure revision of hupehensis saponin F and G and characterization of new trace triterpenoid saponins from Anemone hupehensis by tandem electrospray ionization mass spectrometry. Carbohydrate research 2012; 353:49-56. DOI: 10.1016/j.carres.2012.03.020.
Li K, Wohlford-Lenane C, Perlman S, et al. Middle East Respiratory Syndrome Coronavirus Causes Multiple Organ Damage and Lethal Disease in Mice Transgenic for Human Dipeptidyl Peptidase 4. J Infect Dis 2016; 213(5):712-22. DOI: 10.1093/infdis/jiv499.
Mathieu C, Augusto M T, Niewiesk S, et al. Broad spectrum antiviral activity for paramyxoviruses is modulated by biophysical properties of fusion inhibitory peptides. Sci Rep 2017; 7:43610. DOI: 10.1038/srep43610.
Mathieu C, Bovier F T, Ferren M, et al. Molecular Features of the Measles Virus Viral Fusion Complex That Favor Infection and Spread in the Brain. mBio 2021:e0079921. DOI: 10.1128/mBio.00799-21.
Mathieu C, Huey D, Jurgens E, et al. Prevention of measles virus infection by intranasal delivery of fusion inhibitor peptides. J Virol 2015; 89(2):1143-55. DOI: 10.1128/JVI.02417-14.
Mathieu C, Porotto M, Figueira T N, Horvat B, Moscona A. Fusion Inhibitory Lipopeptides Engineered for Prophylaxis of Nipah Virus in Primates. J Infect Dis 2018; 218(2):218-227. DOI: 10.1093/infdis/jiy152.
McCray P B, Jr., Pewe L, Wohlford-Lenane C, et al. Lethal infection of K18-hACE2 mice infected with severe acute respiratory syndrome coronavirus. J Virol 2007; 81(2):813-21. DOI: 10.1128/JVI.02012-06.
Moscona A, Peluso R W. Relative affinity of the human parainfluenza virus type 3 hemagglutinin-neuraminidase for sialic acid correlates with virus-induced fusion activity. J Virol 1993; 67(11):6463-8.
Moscona A, Porotto M, Palmer S, et al. A recombinant sialidase fusion protein effectively inhibits human parainfluenza viral infection in vitro and in vivo. J Infect Dis 2010; 202(2):234-41. DOI: 10.1086/653621.
Murrell M, Greengard O, Porotto M, Poltoratskaia N, Moscona A. A single amino acid alteration in the human parainfluenza virus type 3 HN confers resistance to both binding inhibition and neuraminidase inhibition by the antiviral agent 4-GU-DANA (Zanamivir). Journal of Virology 2001; 75:6310-6320.
Murrell M, Porotto M, Weber T, Greengard O, Moscona A. Mutations in human parainfluenza virus type 3 HN causing increased receptor binding activity and resistance to the transition state sialic acid analog 4-GU-DANA (zanamivir). J Virol 2003; 77:309-317.
Navaratnarajah C K, Generous A R, Yousaf I, Cattaneo R. Receptor-mediated cell entry of paramyxoviruses: Mechanisms, and consequences for tropism and pathogenesis. The Journal of biological chemistry 2020; 295(9):2771-2786. (In eng). DOI: 10.1074/jbc.REV119.009961.
Netland J, Ferraro D, Pewe L, Olivares H, Gallagher T, Perlman S. Enhancement of murine coronavirus replication by severe acute respiratory syndrome coronavirus protein 6 requires the N-terminal hydrophobic region but not C-terminal sorting motifs. J Virol 2007; 81(20):11520-5. DOI: 10.1128/JVI.01308-07.
Osbourn A, Goss R J, Field R A. The saponins: polar isoprenoids with important and diverse biological activities. Natural product reports 2011; 28(7):1261-8. DOI: 10.1039/c1 np00015b.
Outlaw V K, Bottom-Tanzer S, Kreitler D F, Gellman S H, Porotto M, Moscona A. Dual Inhibition of Human Parainfluenza Type 3 and Respiratory Syncytial Virus Infectivity with a Single Agent. J Am Chem Soc 2019; 141(32):12648-12656. DOI: 10.1021/jacs.9b04615.
Outlaw V K, Bovier F T, Mears M C, et al. Inhibition of Coronavirus Entry In Vitro and Ex Vivo by a Lipid-Conjugated Peptide Derived from the SARS-CoV-2 Spike Glycoprotein HRC Domain. mBio 2020; 11(5). DOI: 10.1128/mBio.01935-20.
Outlaw V K, Cheloha R W, Jurgens E M, et al. Engineering Protease-Resistant Peptides to Inhibit Human Parainfluenza Viral Respiratory Infection. J Am Chem Soc 2021; 143(15):5958-5966. DOI: 10.1021/jacs.1c01565.
Ouyang K, Chen L, Sun H, Du J, Shi M. Screening and appraisal for immunological adjuvant-active fractions from Platycodon grandiflorum total saponins. Immunopharmacology and immunotoxicology 2012; 34(1):126-34. DOI: 10.3109/08923973.2011.586704.
Palermo L M, Uppal M, Skrabanek L, et al. Features of Circulating Parainfluenza Virus Required for Growth in Human Airway. mBio 2016; 7(2):e00235. DOI: 10.1128/mBio.00235-16.
Palmer S G, DeVito I, Jenkins S G, Niewiesk S, Porotto M, Moscona A. Circulating clinical strains of human parainfluenza virus reveal viral entry requirements for in vivo infection. J Virol 2014; 88(22):13495-502. DOI: 10.1128/JVI.01965-14.
Palmer S G, Porotto M, Palermo L M, Cunha L F, Greengard O, Moscona A. Adaptation of human parainfluenza virus to airway epithelium reveals fusion properties required for growth in host tissue. mBio 2012; 3(3):e00137-12. (In eng). DOI: 10.1128/mBio.00137-12.
Park J E, Gallagher T. Lipidation increases antiviral activities of coronavirus fusion-inhibiting peptides. Virology 2017; 511:9-18. DOI: 10.1016/j.virol.2017.07.033.
Park J E, Li K, Barlan A, et al. Proteolytic processing of Middle East respiratory syndrome coronavirus spikes expands virus tropism. Proc Natl Acad Sci USA 2016; 113(43):12262-12267. DOI: 10.1073/pnas.1608147113.
Pessi A, Langella A, Capito E, et al. A general strategy to endow natural fusion-protein-derived peptides with potent antiviral activity. PLoS One 2012; 7(5):e36833. DOI: 10.1371/journal.pone.0036833.
Pompei R, Flore O, Marccialis M A, Pani A, Loddo B. Glycyrrhizic acid inhibits virus growth and inactivates virus particles. Nature 1979; 281(5733):689-90
Porotto M, Carta P, Deng Y, et al. Molecular determinants of antiviral potency of paramyxovirus entry inhibitors. J Virol 2007; 81(19):10567-74. DOI: 10.1128/JVI.01181-07.
Porotto M, Ferren M, Chen Y W, et al. Authentic Modeling of Human Respiratory Virus Infection in Human Pluripotent Stem Cell-Derived Lung Organoids. mBio 2019; 10(3). DOI: 10.1128/mBio.00723-19.
Porotto M, Rockx B, Yokoyama C C, et al. Inhibition of Nipah virus infection in vivo: targeting an early stage of paramyxovirus fusion activation during viral entry. PLoS Pathog 2010; 6(10):e1001168. DOI: 10.1371/journal.ppat.1001168.
Porotto M, Yokoyama C C, Palermo L M, et al. Viral entry inhibitors targeted to the membrane site of action. Journal of virology 2010; 84(13):6760-8. (In Eng). DOI: 10.1128/JVI.00135-10.
Pruijssers A J, Denison M R. Nucleoside analogues for the treatment of coronavirus infections. Curr Opin Virol 2019; 35:57-62. DOI: 10.1016/j.coviro.2019.04.002.
Sando T, Hayashi T, Takeda T, et al. Histochemical study of detailed laticifer structure and rubber biosynthesis-related protein localization in Hevea brasiliensis using spectral confocal laser scanning microscopy. Planta 2009; 230(1):215-25. DOI: 10.1007/s00425-009-0936-0.
Sheahan T P, Sims A C, Zhou S, et al. An orally bioavailable broad-spectrum antiviral inhibits SARS-CoV-2 in human airway epithelial cell cultures and multiple coronaviruses in mice. Sci Transl Med 2020; 12(541). DOI: 10.1126/scitranslmed.abb5883.
Sun H, Chen L, Wang J, Wang K, Zhou J. Structure-function relationship of the saponins from the roots of Platycodon grandiflorum for hemolytic and adjuvant activity. International immunopharmacology 2011; 11(12):2047-56. DOI: 10.1016/j.intimp.2011.08.018.
Tangudu C, Olivares H, Netland J, Perlman S, Gallagher T. Severe acute respiratory syndrome coronavirus protein 6 accelerates murine coronavirus infections. J Virol 2007; 81(3):1220-9. DOI: 10.1128/JVI.01515-06.
Thakkar V D, Cox R M, Sawatsky B, et al. The Unstructured Paramyxovirus Nucleocapsid Protein Tail Domain Modulates Viral Pathogenesis through Regulation of Transcriptase Activity. J Virol 2018; 92(8). DOI: 10.1128/JVI.02064-17.
Thompson Cl, Barclay W S, Zambon M C, Pickles R J. Infection of human airway epithelium by human and avian strains of influenza A virus. J Virol 2006; 80(16):8060-8. (In eng)
Wang R, Simoneau C R, Kulsuptrakul J, et al. Genetic Screens Identify Host Factors for SARS-CoV-2 and Common Cold Coronaviruses. Cell 2021; 184(1):106-119 e14. DOI: 10.1016/j.cell.2020.12.004.
Welsch J C, Talekar A, Mathieu C, et al. Fatal measles virus infection prevented by brain-penetrant fusion inhibitors. J Virol 2013; 87(24):13785-94. DOI: 10.1128/JVI.02436-13.
White J M, Delos S E, Brecher M, Schornberg K. Structures and mechanisms of viral membrane fusion proteins: multiple variations on a common theme. Critical reviews in biochemistry and molecular biology 2008; 43(3):189-219.
Xia S, Liu M, Wang C, et al. Inhibition of SARS-CoV-2 (previously 2019-nCoV) infection by a highly potent pan-coronavirus fusion inhibitor targeting its spike protein that harbors a high capacity to mediate membrane fusion. Cell Res 2020; 30(4):343-355. DOI: 10.1038/s41422-020-0305-x.
Xia S, Yan L, Xu W, et al. A pan-coronavirus fusion inhibitor targeting the HR1 domain of human coronavirus spike. Sci Adv 2019; 5(4):eaav4580. DOI: 10.1126/sciadv.aav4580.
Xie X, Muruato A, Lokugamage K G, et al. An Infectious cDNA Clone of SARS-CoV-2. Cell Host Microbe 2020; 27(5):841-848 e3. DOI: 10.1016/j.chom.2020.04.004.
Yoon J J, Toots M, Lee S, et al. Orally Efficacious Broad-Spectrum Ribonucleoside Analog Inhibitor of Influenza and Respiratory Syncytial Viruses. Antimicrob Agents Chemother 2018; 62(8). DOI: 10.1128/AAC.00766-18.
Yu D, Sakurai Y, Chen C H, et al. Anti-AIDS agents 69. Moronic acid and other triterpene derivatives as novel potent anti-HIV agents. Journal of medicinal chemistry 2006; 49(18):5462-9. DOI: 10.1021/jm0601912.
Zhu Y, Yu D, Yan H, Chong H, He Y. Design of Potent Membrane Fusion Inhibitors against SARS-CoV-2, an Emerging Coronavirus with High Fusogenic Activity. J Virol 2020; 94(14). DOI: 10.1128/JVI.00635-20.

1. A method for
(a) treating a viral disease other than COVID-19 in a subject;
(b) inhibiting an interaction between a viral protein other than SARS-CoV-2 spike protein and cellular ACE2 in a subject;
(c) reducing the severity of a viral infection other than SARS-CoV-2 in a subject;
(d) reducing viral load in a subject of a subject infected with or exposed to a virus other than SARS-CoV-2;
(e) preventing disease progression in a subject infected by a virus other than SARS-CoV-2;
(f) reducing the duration of an infection by a virus other than SARS-CoV-2;
(g) reducing the risk of severe disease or death caused by or ancillary to a viral infection in a subject infected with a virus other than SARS-CoV-2;
(h) treating a viral infection in a subject infected by a virus other than SARS-CoV-2; and/or
(i) preventing a viral infection in a subject exposed or at risk of exposure to a virus other than SARS-CoV-2;
the method comprising administering a lipopeptide comprising at least one peptide unit comprising or consisting of an amino acid sequence having at least 80% identity to SEQ ID NO:1 to a subject in need thereof.

2. The method of claim 1, wherein the subject is a mammal.

3. The method of claim 1, wherein the virus expresses a protein that utilizes the ACE2 receptor for cellular entry.

4. The method of claim 1, wherein the virus is a coronavirus.

5. The method of claim 4, wherein the coronavirus is an alphacoronavirus or an embecovirus.

6. (canceled)

7. The method of claim 1, wherein the virus is a measles virus, a Nipah virus, an Ebola virus, a parainfluenza virus, or an influenza virus.

8-15. (canceled)

16. The method of claim 1, wherein the at least one peptide unit comprises or consists of an amino acid sequence having 100% identity to SEQ ID NO:1.

17. The method claim 1, wherein the at least one peptide unit comprises from 1 to 7 β-amino acids.

18. The method of claim 1, wherein the at least one peptide unit further comprises a GSGSGC unit.

19. (canceled)

20. The method of claim 1, wherein the lipopeptide further comprises at least one lipid unit.

21. The method of claim 20, wherein the at least one lipid unit comprises cholesterol, tocopherol, or palmitate.

22. (canceled)

23. The method of claim 1, wherein the lipopeptide further comprises at least one linker unit.

24. The method of claim 23, wherein the at least one linker unit comprises polyethylene glycol (PEG).

25. The method of claim 24, wherein the at least one linker unit comprises or consists of PEG4, PEG11, PEG24, or PEG28.

26-29. (canceled)

30. The method of claim 1, wherein the lipopeptide has the structure of formula A:

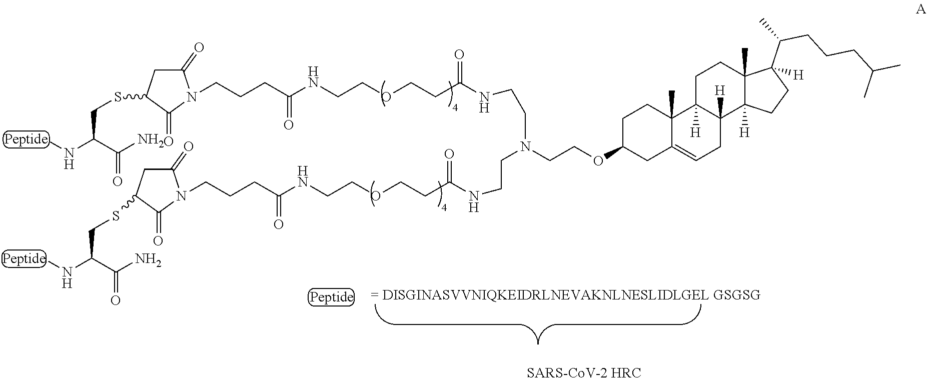

31-32. (canceled)

33. The method of claim 1, wherein the lipopeptide is formulated in a dosage form selected from the group consisting of: a liquid, a paste, a bar, a cake, a powder, a granulate, a chewable, a tablet, a capsule, a lozenge, a fast-melting tablet or wafer, and a sublingual tablet.

34. (canceled)

35. The method of claim 1, wherein the lipopeptide is administered to the subject's airway.

36-38. (canceled)

39. The method of claim 1, wherein the lipopeptide is administered at least once daily or twice daily.

40-42. (canceled)

43. The method of claim 39, wherein the lipopeptide is administered for a period of at least one day, at least two days, or at least 3 days and/or over a treatment period of up to a week or up to two weeks.

44-45. (canceled)

46. The method of claim 1, wherein the method is:
(A) for (b) and/or (i) and the lipopeptide is administered at least once before the subject is exposed to the virus;
(B) for (b) and/or (i) and the lipopeptide is administered at least twice before the subject is exposed to the virus;
(C) for one or more of (a) to (h) and the lipopeptide is administered at least once before the subject develops symptoms of viral disease;
(D) for one or more of (a) to (h) and the lipopeptide is administered at least twice before the subject develops symptoms of viral disease;
(E) for one or more of (a) to (h) and the lipopeptide is administered at least once after the onset of symptoms of viral disease; or
(F) for one or more of (a) to (h) and the lipopeptide is administered at least twice after the onset of symptoms of viral disease.

47-51. (canceled)


SEQUENCE LISTING (6 sequences)

SEQ ID NO: 1 - protein, 36 aa, synthetic construct
DISGINASVVNIQKEIDRLNEVAKNLNESLIDLQEL

SEQ ID NO: 2 - protein, 36 aa, synthetic construct
DISQINASVVNIEYEIKKLEEVAKKLEESLIDLQEL

SEQ ID NO: 3 - protein, 36 aa, synthetic construct
SIDQINATFVDIEYEIKKLEEVAKKLEESYIDLKEL

SEQ ID NO: 4 - protein, 36 aa, synthetic construct
DISQINASVVNIEYEIZKLEUVAZKLEUSLIDLQEL

SEQ ID NO: 5 - protein, 36 aa, synthetic construct
SLDQINVTFLDLEYEMKKLEEAIKKLEESYIDLKEL

SEQ ID NO: 6 - protein, 36 aa, synthetic construct
SLTQINTTLLDLTYEMLSLQQVVKALNESYIDLKEL